(19) United States
(12) Patent Application Publication
(10) Pub. No.: US 2006/0194240 A1
(43) Pub. Date: Aug. 31, 2006

(54) Compositions and methods of detecting an analyte by using a nucleic acid hybridization switch probe

(75) Inventors: Lyle J. Arnold JR., Poway, CA (US); Lizhong Dai, San Diego, CA (US); Steven T. Brentano, Santee, CA (US); James Russell, Vista, CA (US)

Correspondence Address: GEN PROBE INCORPORATED, 10210 GENETIC CENTER DRIVE, SAN DIEGO, CA, 92121, US

(73) Assignee: Gen-Probe Incorporated, San Diego, CA (US)

(21) Appl. No.: 11/363,848

(22) Filed: Feb. 28, 2006

Related U.S. Application Data
(60) Provisional application No. 60/657,523, filed on Feb. 28, 2005.

(51) Int. Cl. C12Q 1/68; C07H 21/04

(52) U.S. Cl. 435/6; 536/24.3

(57) ABSTRACT

Compositions are described for detecting binding of an analyte to a binding partner attached to a nucleic acid hybridization switch probe that includes first and second arm sequences and a support sequence that is at least partially complementary to both arm sequences, allowing the probe under hybridization conditions to form a first conformation in the absence of the analyte and to form a second conformation in the presence of the analyte, and a label associated with the probe that produces a signal that indicates the conformation of the probe. Methods are described for detecting an analyte that forms a specific binding pair with the binding partner attached to the hybridization switch probe, thereby changing the probe from a first to a second conformation that results in a detectable signal that indicates the presence of the analyte in the sample.

### RELATED APPLICATION

This application claims the benefit of U.S. provisional application no. 601657,523, filed Feb. 28, 2005, under 35 U.S.C. 119(e), the contents of which are hereby incorporated by reference herein.

### FIELD OF THE INVENTION

This invention relates to detection of chemical or biochemical molecules in a sample, and specifically relates to compositions and assays for detecting an analyte by using a nucleic acid oligomer probe that includes a first member of a specific binding pair that binds specifically to the analyte, and complementary nucleic acid sequences that form a hybridization complex, whereby detecting a conformational change in the oligomer probe indicates the presence of the analyte in a sample.

### BACKGROUND OF THE INVENTION

Detection of a chemical or biochemical molecule is used in many applications, such as in diagnostic assays, environmental and food testing, forensic methods to detect chemical, biochemical, or biological evidence, epidemiological assays to identify or characterize pathological or infectious agents, and the like. Such assays often detect a binding pair complex made up of one member of a binding pair and a second member of the binding pair that is the analyte to be detected. Known types of binding pairs include an antigen or ligand with its antibody or Fab fragment, a hormone or other cell-signaling molecule (e.g., neurotransmitter or interleukin) with its cognate receptor, a drug with its receptor, an enzyme with its substrate or cofactor, and complementary nucleic acid sequences that form hybridization complexes. As illustrated by these examples, a member of a binding pair may be a chemical or biochemical compound, complex, or aggregate (e.g., cell fragment or organelle).

Methods of detecting analytes that are members of binding pairs are known. Such methods may rely on formation, or inhibition of formation, of a binding pair complex and detection of a signal associated with such binding pair complex formation or inhibition. Assays that detect binding pair complexes include immunoprecipitation assays, radioimmunoassays (RIA), enzyme linked immunosorbent assays (ELISA), immuno-polymerase chain reaction assays (iPCR), nucleic acid hybridization assays (e.g., Southern blots or biochip assay), and protein binding assays (e.g., Western blot). Such assays often produce a visible or detectable precipitate, gel, aggregate, or a signal associated with the binding pair complex. In one general assay format, a detectable signal is produced directly or indirectly from a label associated with the binding pair complex that includes the target analyte. In another general assay format, a signal is inhibited when the target analyte is present and inhibits formation of a detectable binding pair complex that produces a signal. Such assays may rely on a variety of labels to produce detectable signals under appropriate conditions, e.g., radionuclides, enzymes, dyes, chromophores, fluorophores, or luminescent compounds.

Many applications of analytical assays require detection of small quantities of a target analyte present in a sample and, hence, methods and components have been developed to increase assay sensitivity. Examples include use of monoclonal antibodies, Fab fragments, or synthetic constructs that have a higher affinity for the target antigen or ligand than polyclonal antibodies, and use of enzymatic turnover in an ELISA. Other examples include amplification of target or probe nucleic acid sequences (e.g., U.S. Pat. No. 4,683,195, Mullis et al.; U.S. Pat. No. 4,786,600, Kramer et al.; U.S. Pat. No. 5,130,238, Malek et al.; U.S. Pat. No. 5,409,818, Davey et al.; U.S. Pat. No. 5,422,252, Walker et al.; U.S. Pat. No. 5,215,899, Dattagupta; U.S. Pat. No. 6,087,133, Dattagupta et al.; U.S. Pat. No. 5,827,649, Rose et al.; U.S. Pat. No. 5,399,491, Kacian et al.; U.S. Pat. Nos. 5,714,320 and 6,077,668, Kool), and a combination of immunocomplex formation and nucleic acid amplification in an immuno-PCR (iPCR) reaction (e.g., WO 2004072301, McCreavy et al.). Signal amplification may be achieved by making large aggregates of hybridization complexes that include target nucleic acids (e.g., U.S. Pat. Nos. 5,710,264, 5,849,481, and 5,124,246, Urdea et al.; U.S. Pat. No. 6,221,581, Engelhardt et al.).

Many detection methods require that the unbound label be separated from the binding pair complex before the detection step is performed because unbound label produces a signal that cannot be distinguished from the signal produced from the label associated with the analyte-containing binding pair complex. That is, the presence of the target analyte cannot be detected unless unbound labeled components are separated from the reaction mixture because the signal from the unbound labeled components masks the signal from the label associated with the binding pair complex.

A homogeneous assay format allows detection of the signal from the label associated with the target analyte without removal of the unbound label. Such systems, however, may have reduced sensitivity because a relatively high background signal may be produced from the retained unbound label compared to systems in which the unbound label is removed. A homogeneous system used to reduce background and increase assay sensitivity, referred to as a “homogeneous protection assay” (HPA), includes a binding partner of the analyte, labeled with a substance that exhibits detectable changes in stability when the analyte binds the binding partner (e.g., U.S. Pat. Nos. 5,283,174 and 5,639,604, Arnold et al.).

Known systems of detecting nucleic acids in hybridization complexes use nucleic acid probes that preferentially produce a signal when the probe is hybridized to the probe's nucleic acid target sequence. Such probes include a probe sequence surrounded by switch sequences that are complementary to each other and have been referred to as “molecular switch” or “molecular beacon” probes (e.g., U.S. Pat. Nos. 5,118,801 and 5,312,728, Lizardi et al., U.S. Pat. Nos. 5,925,517 and 6,150,097, Tyagi et al.). Such probes generally include a label (e.g., a fluorophore) on one switch sequence and an inhibitor compound (e.g., chromophore) on the other switch sequence to inhibit or quench the signal from the label when the label and inhibitor compounds are in close proximity, as occurs when a hairpin probe is in a closed conformation. When the probe sequence hybridizes to its target nucleic acid, the probe switches to an open conformation that separates the label and inhibitor compounds, thus producing a detectable signal. Another system, referred to as a “molecular torch” probe includes a target binding domain, a target closing domain, and a joining region, in which the target binding domain forms a more stable hybrid with the target sequence than with the target closing domain under the same hybridization conditions, thus producing a detectable signal when the target sequence is present (U.S. Pat. No. 6,361,945, Becker et al.).

### SUMMARY OF THE INVENTION

One aspect of the invention is a hybridization switch probe (HSP) specific for detection of an analyte, that includes a first nucleic acid arm sequence; a second nucleic acid arm sequence that is different from the first nucleic acid arm sequence; a nucleic acid support sequence that is at least partially complementary to the first nucleic acid arm sequence and at least partially complementary to the second nucleic acid arm sequence, whereby under hybridization conditions the support sequence forms a hybridization duplex with either the first nucleic acid arm sequence thereby forming a first HSP conformation, or the second nucleic acid arm sequence thereby forming a second HSP conformation; a label that produces a signal that indicates the conformation of the hybridization switch probe, and a binding pair member that forms a specific binding pair complex with the analyte, wherein the specific binding pair complex produces a conformational change in the hybridization switch probe that results in a detectable signal. In one embodiment of the hybridization switch probe, the first arm sequence is shorter than the second arm sequence. In another embodiment, the label produces a signal that is detectable in a homogeneous assay system. In one embodiment, the label is a portion of the HSP nucleic acid, whereas in another embodiment, the label is a separate moiety joined directly or indirectly to the HSP. In some preferred embodiments, the label is selected from the group consisting of: a HSP nucleic acid sequence that binds a separate nucleic acid probe sequence, a HSP nucleic acid sequence that serves as a primer in a nucleic acid amplification reaction, a HSP nucleic acid sequence that serves as a template in a nucleic acid amplification reaction, and an aptamer. In other preferred embodiments, the label is selected from the group consisting of a radionuclide, a ligand, an enzyme, an enzyme substrate, an enzyme cofactor, a reactive group, a chromophore, a particle, a bioluminescent compound, a phosphorescent compound, a chemiluminescent compound, and a fluorophore. A preferred embodiment includes a label that is a chemiluminescent compound attached to either the first arm sequence or the second arm sequence. In one embodiment the label is a fluorophore attached to the first arm sequence and the support sequence includes a quencher compound that is in close proximity to the fluorophore when the first arm sequence and the support sequence form a hybridization duplex. In another embodiment, the label is a fluorophore attached to the second arm sequence and the support sequence includes a quencher compound that is in close proximity to the fluorophore when the second arm sequence and the support sequence form a hybridization duplex. In another embodiment, the label is a fluorophore attached to the support sequence and the first arm sequence includes a quencher compound that is in close proximity to the fluorophore when the first arm sequence and the support sequence form a hybridization duplex. In another embodiment, the label is a fluorophore attached to the support sequence and the second arm sequence includes a quencher compound that is in close proximity to the fluorophore when the second arm sequence and the support sequence form a hybridization duplex. In one embodiment, the first arm sequence is joined to the support sequence by a linking element and the second arm sequence is joined to the support sequence by a linking element. In some embodiments, the binding pair member that forms a specific binding pair complex with the analyte is an aptamer. In some hybridization switch probes, the detectable signal is an amplified nucleic acid that is produced by use of a portion of the HSP participating in a nucleic acid amplification reaction.

Another aspect of the invention is a kit that includes a hybridization switch probe made up of a first nucleic acid arm sequence; a second nucleic acid arm sequence that is different from the first nucleic acid arm sequence; a nucleic acid support sequence that is at least partially complementary to the first nucleic acid arm sequence and to the second nucleic acid arm sequence, whereby under hybridization conditions the support sequence forms a hybridization duplex with the first nucleic acid arm sequence to form a first conformation of the hybridization switch probe, or with the second nucleic acid arm sequence to form a second conformation of the hybridization switch probe; a label that produces a signal that indicates the conformation of the hybridization switch probe; and a binding pair member that forms a specific binding pair complex with an analyte detected by the hybridization switch probe, wherein the specific binding pair complex produces a conformational change in the hybridization switch probe that results in a detectable signal from the label. Embodiments of the kit may further include one or more reagents for preparation of a sample containing the analyte, to promote binding of the analyte and the binding pair member, to treat the label to produce a detectable signal, or to be used in a nucleic acid amplification reaction that amplifies a nucleic acid sequence by using a portion of the HSP.

Another aspect of the invention is a method of detecting an analyte in a sample, that includes the steps of forming a reaction mixture comprising a sample containing an analyte and a hybridization switch probe specific for the analyte, wherein the hybridization switch probe is made up of a first nucleic acid arm sequence, a second nucleic acid arm sequence that is different from the first nucleic acid arm sequence, a nucleic acid support sequence that is at least partially complementary to the first nucleic acid arm sequence and to the second nucleic acid arm sequence, a label that produces a detectable signal, and a binding pair member that binds the analyte to form a specific binding pair complex that produces a conformational change in the hybridization switch probe, and wherein the hybridization switch probe is in a first HSP conformation in which one arm sequence is in a hybridization duplex with the support sequence; binding the analyte to the binding pair member, thereby forming a specific binding pair complex on the hybridization switch probe; producing a conformational change from the first HSP conformation to a second HSP conformation resulting from formation of the specific binding pair complex; and detecting a signal change from the label that indicates the conformational change, thereby indicating the presence of the analyte in the sample. In one embodiment, the first arm sequence of the HSP has an attached label, the second arm sequence has an attached binding pair member, and the first HSP conformation includes a hybridization duplex made up of the second arm sequence and the support sequence which is destabilized when the specific binding pair complex is formed, thereby changing the HSP to the second HSP conformation that includes a hybridization duplex made up of the first arm sequence and the support sequence. In another embodiment, the second arm sequence of the HSP has an attached label, the first arm sequence has an attached binding pair member, and the first HSP conformation includes a hybridization duplex made up of the first arm sequence and the support sequence which is destabilized when the specific binding pair complex is formed, thereby changing the hybridization switch probe to the second HSP conformation that includes a hybridization duplex made up of the second arm sequence and the support sequence. In another embodiment, one arm sequence of the hybridization switch probe is a labeled arm sequence that has both an attached label and an attached binding pair member, and the first HSP conformation includes a hybridization duplex made up of the labeled arm sequence and the support sequence which is destabilized when the specific binding pair complex is formed, thereby changing the hybridization switch probe to the second HSP conformation in which the labeled arm sequence is not hybridized to the support sequence. In another embodiment, the analyte is a ligand that binds specifically to the binding pair member and both the binding pair member and analyte have known chemical or biochemical structures. In a different embodiment, the analyte is a ligand that binds specifically to the binding pair member and either the ligand or the binding pair member has an unknown chemical or biochemical structure. In another embodiment, the binding pair member is a portion of a nucleic acid sequence in the hybridization switch probe. In a preferred embodiment, the binding pair member is an aptamer. In one embodiment, the detecting step detects an increase in a detectable signal to indicate the presence of the analyte in the sample, whereas in another embodiment, the detecting step detects a decrease in a detectable signal to indicate the presence of the analyte in the sample. In one embodiment, the detecting step detects a signal resulting from in vitro amplification of a nucleic acid sequence present in the HSP. In another embodiment, the detecting step detects a signal resulting from using a portion of the hybridization switch probe in the second HSP conformation as a primer or template in an in vitro nucleic acid amplification reaction. In another embodiment, the detecting step detects a signal resulting from using a portion of the hybridization switch probe in the first HSP conformation as a primer or template in an in vitro nucleic acid amplification reaction. In one embodiment, the detecting step detects a signal resulting from in vitro amplification of a sequence that is only amplified when the hybridization switch probe is in the second HSP conformation. In another embodiment, the detecting step detects a signal resulting from in vitro amplification of a sequence that is only amplified when the hybridization switch probe is in the first HSP conformation. In preferred embodiments, the detecting step is performed in a homogeneous format.

The accompanying drawings, which constitute a part of the specification, illustrate aspects of some embodiments of the invention. These drawings, together with the description, serve to explain and illustrate the principles of the invention.

### BRIEF DESCRIPTION OF THE DRAWINGS

FIG. 1A to FIG. 1D are schematic drawings of different embodiments of a hybridization switch probe (HSP). FIG. 1A illustrates a HSP made of two complementary nucleic acid sequences present in separate strands that are joined in an intermolecular hybridization duplex by standard base pairing that occurs under hybridization conditions, where a first strand (1) has an attached label (L) and a second strand (2) has an attached member of a binding pair (M₁) specific for the analyte to be detected. FIG. 1B illustrates a HSP made of two complementary nucleic acid sequences (1, 2) that are covalently joined by a linker element (LE), and the two complementary sequences are joined in an intramolecular hybridization duplex by base pairing, in which the first sequence (1) has an attached label (L) and the second sequence (2) has an attached member of a binding pair (M₁) specific for the analyte. FIG. 1C illustrates a HSP made of three nucleic acid sequences (1, 2, 3) that are covalently joined by linker elements (LE), in which a first arm sequence (1) has an attached label (L), the second arm sequence (2) has an attached member of a binding pair (M₁) specific for the analyte, and an intervening support sequence (3) is at least partially complementary to both arm sequences (1 and 2), as shown by the hybridization duplex formed between sequences 2 and 3. FIG. 1D illustrates a HSP made of three nucleic acid sequences (1, 2, 3) that are covalently joined by linker elements (LE), in which a first arm sequence (1) has an attached label (L), a second arm sequence (2) has an attached member of a binding pair (M₁) specific for the analyte, and a terminal support sequence (3) is at least partially complementary to both arm sequences (1 and 2), as shown by the hybridization duplex formed between sequences 2 and 3.

FIG. 2 is a schematic diagram of a hybridization switch probe-based assay in which the HSP includes a first arm sequence (1) with an attached acridinium ester label (AE) and a second arm sequence (2) with an attached binding pair member (M₁) specific for the analyte (M₂). In the upper portion, in the absence of analyte, the second arm sequence (2) is hybridized to a portion of the support sequence (3) of the HSP and the first arm (1) is a substantially single-stranded portion of the HSP. In the lower portion, in the presence of analyte, the analyte (M₂) is attached to the binding pair member (M₁) which destabilizes the duplex between the second arm sequence (2) and the support sequence (3), allowing formation of a hybridization duplex made up of the first arm sequence (1) and the support sequence (3).

FIG. 3 is a schematic diagram of a hybridization switch probe-based assay in which the HSP includes a first arm sequence (1) with an attached label (L), joined by a linker element (LE) to the second sequence arm sequence (2) with an attached binding pair member (M₁) specific for its analyte (M₂), joined by a linker element (LE) to the support sequence (3). The analyte (M₂) is a specific binding partner for the HSP binding pair member (M₁). The upper portion shows the HSP elements in a linear configuration; the middle portion shows the HSP in the absence of analyte with sequences 2 and 3 in a hybridization duplex; and the lower portion shows the HSP in the presence of analyte with sequences 1 and 3 in a hybridization duplex. In the absence of analyte (M₂), the hybridization duplex made up of sequences 2 and 3 is favored, whereas in the presence of the analyte, a conformational change in the HSP results from the analyte (M₂) binding to the binding pair member (M₁) to form a binding pair complex (BPC) that destabilizes the duplex of sequences 2 and 3, thus favoring formation of a hybridization duplex made up of sequences 1 and 3.

FIG. 4A is a schematic diagram of a hybridization switch probe-based assay that uses a HSP that includes a first arm sequence (1) labeled with a fluorophore (F), joined by a linker element (LE) to a support sequence (3) with an attached quencher compound (Q), joined by a linker element (LE) to the second arm sequence (2) with an attached binding pair member (M₁) specific for the analyte (M₂). In the upper portion, in the absence of the analyte, the HSP is in a first conformation in which the second arm sequence (2) is hybridized to a portion of the support sequence (3) and the fluorophore (F) is distant from the quencher (Q), allowing fluorescence. In the lower portion, in the presence of the analyte, the HSP is in a second conformation, which results from the analyte (M₂) binding to the binding pair member (M₁) to form a binding pair complex (BPC) that destabilizes the duplex between the second arm (2) and support (3) sequences, and allowing the first arm (1) and support (3) sequences to form a hybridization duplex which brings the fluorophore (F) and quencher (Q) into close proximity to decrease fluorescence.

FIG. 4B is a schematic diagram of a hybridization switch probe-based assay that uses a HSP that includes a first arm sequence (1) joined by a linker element (LE) to a support sequence (3) with an attached quencher compound (Q), joined by a linker element (LE) to the second arm sequence (2) with an attached binding pair member (M₁) specific for the analyte (M₂) and a fluorophore label (F). In the upper portion, in the absence of the analyte, the HSP is in a first conformation in which the second arm sequence (2) is hybridized to a portion of the support sequence (3) and the fluorophore (F) and quencher (Q) are in close proximity which reduces fluorescence. In the lower portion, in the presence of the analyte, the HSP is in a second conformation, which results from the analyte (M₂) binding to the binding pair member (M₁) to form a specific binding pair complex (BPC) that destabilizes the duplex between the second arm (2) and support (3) sequences and separates the fluorophore (F) and quencher (Q) to increase fluorescence, and allows formation of a hybridization duplex made up of the first arm sequence (1) and support sequence (3).

FIG. 5 is a schematic diagram of a generic HSP-based assay in which the HSP includes a binding pair member (M₁) specific for the analyte (M₂) and a label. In the upper portion, in the absence of the analyte, the HSP is in a first conformation in which the label is in an inactive state, whereas in the lower portion, in the presence of the analyte, the analyte (M₂) and its binding pair member (M₁) form a specific binding pair complex (BPC), thus changing the HSP to a second conformation in which the label is in an active state.

FIG. 6 is a graphic display of a titration of an AE-labeled HSP with attached biotin (HSP 15-13, SEQ ID NO:12) by using an analyte, streptavidin, that forms a specific binding pair with biotin, showing the streptavidin amounts (fmol) present in the reaction mixture on the X-axis and the detected signal (relative light units or “RLU”) on the Y-axis.

FIG. 7 is a graphic display of a titration of an AE-labeled HSP with attached biotin (HSP 16-14 at 40 fmol; SEQ ID NO:15) by using an analyte, streptavidin, that forms a specific binding pair with biotin, showing the streptavidin amounts (fmol) present in the reaction mixture on the X-axis and the detected signal (RLU) on the Y-axis.

FIG. 8 is a graphic display of a titration of an AE-labeled HSP with attached biotin (HSP 16-14 at 2 fmol) by using an analyte, streptavidin, that forms a specific binding pair with biotin, showing the streptavidin amounts (fmol) present in the reaction mixture on the X-axis and the detected signal (RLU) on the Y-axis.

FIG. 9 is a graphic display of a competition titration assay of an AE-labeled HSP with attached biotin (HSP 16-14) by using an analyte, streptavidin, and free biotin in solution as the competitor for the analyte that forms a specific binding pair with the biotin attached to the HSP, showing the competitor biotin amounts (fmol) present in the reaction mixture on the X-axis and the detected signal (RLU) on the Y-axis.

### DETAILED DESCRIPTION OF THE INVENTION

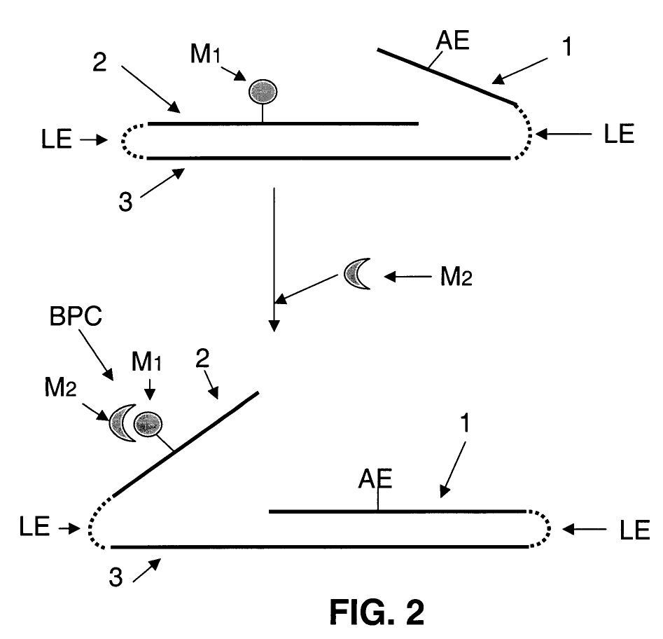

The invention includes methods of detecting analytes by combining nucleic acid hybridization in a hybridization switch probe (HSP) with a specific binding interaction between members of a specific binding pair to induce a conformational change in the hybridization switch probe. The invention also includes a HSP that is a nucleic acid oligomer that includes at least two arm sequences that are complementary to a support sequence of the probe, where a first arm sequence has a label that produces a signal and a second arm sequence has one member of a specific binding pair. Both of the arm sequences are complementary to a portion of the support sequence to favor formation of a hybridization duplex between one of the arm sequences and the support sequence under the appropriate hybridization conditions. In one embodiment, a binding interaction between the analyte and the binding pair member on an arm sequence alters the stability of a hybridization complex between one of the arm sequences and the support sequence of the HSP, resulting in a conformational change in the HSP that results in a change in signal (i.e., production or loss of signal), depending on the label used. For example, as illustrated in the embodiment shown in FIG. 2, a binding interaction between the analyte (M₂) and its specific binding pair partner (M₁) on the second arm (2) destabilizes the duplex of strands 2 and 3, which then favors formation of a hybridization duplex between the labeled arm (1) and the support sequence (3). This conformational change in the HSP stabilizes the acridinium ester (AE) label allowing it to produce a detectable chemiluminescent signal in a homogeneous protection assay when the analyte is present. That is, in the upper portion of FIG. 2, the AE label is susceptible to degradation, whereas in the lower portion, the AE label is protected from hydrolysis, thus allowing a chemiluminescent signal to be detected when analyte is bound to the HSP. In a preferred embodiment, the amount of analyte present in an assay that uses a HSP correlates linearly with the amount of signal detected from the label of the HSP in a homogeneous assay.

To aid in understanding aspects of the invention described herein, some terms used in this description are defined below.

By “sample” is meant any representative part or item to be tested, and generally refers to any liquid, solid or gaseous mixture that may contain the analyte of interest to be detected by using a HSP. For example, a sample may be a water or soil specimen, a portion of foodstuffs, a specimen of biological origin, or components separated from a specimen. A biological sample would include, without limitation, any tissue or material derived from a living or dead human or animal that may contain the target analyte, for example, sputum, peripheral blood, plasma, serum, swab samples taken from a bodily orifice, biopsy specimens, respiratory tissue or exudates, gastrointestinal tissue, urine, feces, semen or other body fluids. A biological sample may be tissue, fluids or materials derived from plants or microorganisms. A biological sample may be treated to physically or mechanically disrupt the material or cell structure, to release intracellular components and other materials into a solution or suspension that is prepared by using standard laboratory methods to make a sample suitable for analysis by using a HSP. A sample may be treated by using standard procedures (e.g., filtration, centrifugation, sedimentation, and the like) to separate components of a specimen into a solution or suspension that is amenable to HSP-based testing.

By “nucleic acid” is meant a multimeric compound comprising nucleosides or nucleoside analogs which have nitrogenous heterocyclic bases, or base analogs, where the nucleosides are linked together by phosphodiester bonds to form a polynucleotide, which includes ribonucleic acid (RNA) and deoxyribonucleic acid (DNA) and analogs thereof. A nucleic acid “backbone” may be made up of a variety of linkages known in the art, including one or more of sugar-phosphodiester linkages, peptide-nucleic acid bonds (PCT Pub. No. WO 95/32305, Hydig-Hielsen et al.), phosphorothioate linkages, methylphosphonate linkages or combinations thereof. Sugar moieties of the nucleic acid may be either ribose or deoxyribose, or similar compounds having known substitutions, e.g., 2′ methoxy or 2′ halide substitutions. The nitrogenous bases may be conventional bases (A, G, C, T, U), known analogs (e.g., inosine or “I”), known derivatives of purine or pyrimidine bases (e.g., N⁴-methyl deoxygaunosine, deaza- or aza-purines and deaza- or aza-pyrimidines, pyrimidine bases having substituent groups at the 5 or 6 position, purine bases having an altered or a replacement substituent at the 2, 6 or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; see U.S. Pat. No. 5,378,825 and WO 93/13121) or “abasic” residues where the backbone includes no nitrogenous base for one or more residues of the polymer (U.S. Pat. No. 5,585,481, Arnold et al.). A nucleic acid may include only conventional sugars, bases and linkages found in RNA and/or DNA, or may include both conventional components and substitutions (e.g., conventional bases linked via a 2′ methoxy backbone, or a nucleic acid containing conventional bases and one or more base analogs). Nucleic acids may be polymers made up of many thousands of bases, or may be oligonucleotides or oligomers that generally are made up of 1000 or fewer bases, and typically are made up of 100 or fewer bases. Oligomers include polymers falling in a size range having a lower limit of about 2 to 5 bases and an upper limit of about 500 to 900 bases, with preferred oligomers in a size range having a lower limit of about 50 bases and an upper limit of about 70 bases, which may be synthesized by using any of a variety of well known enzymatic or chemical methods and purified by using standard laboratory methods, e.g., chromatography.

The backbone composition of a nucleic acid sequence may affect stability of a hybridization complex that includes that sequence. Preferred backbones include sugar-phosphodiester linkages as in conventional RNA or DNA or derivatives thereof, peptide linkages as in peptide nucleic acids, and sugar-phosphodiester linkages in which a sugar group and/or linkage joining the groups is altered relative to standard DNA or RNA. For example, a sequence having one or more 2′-methoxy substituted RNA groups or 2′-fluoro substituted RNA groups may enhance stability of a hybridization complex with a complementary 2′ OH RNA sequence. Other embodiments include linkages with charged groups (e.g., phosphorothioates) or neutral groups (e.g., methylphosphonates) to affect complex stability.

A “probe” refers generally to a nucleic acid oligomer that is used to detect the presence of an analyte in a sample. A hybridization switch probe (HSP) refers to a probe made up of different functional portions, that preferably are covalently linked. Functional portions of a HSP include a first arm sequence that has an attached binding partner specific for the analyte to be detected, a second arm sequence that has an attached label that produces a signal dependent on the conformation of the second arm relative to a support sequence, and a support sequence that contains portions that are complementary to the first arm sequence and to the second arm sequence. The portions of the support sequence that are complementary to the first and second arm sequences are preferably overlapping sequences in the complete support sequence. Thus, under conditions that permit hybridization, one of the arm sequences is favored to hybridize to the support sequence to form a hybridization duplex. The location of a support sequence relative to the arm sequences is not critical, e.g., the support sequence may be an intervening sequence between the arm sequences or the support sequence may be at a 5′ or 3′ terminal location on the oligomer that includes at least one arm sequence. A support sequence may be directly covalently linked to one or both arm sequences or two sequences of an HSP may be linked via a linker element which may be another oligomeric sequence or other chemical component.

An “analyte” or “target analyte” of a probe generally refers to the chemical, biochemical or biological entity of interest in a sample to be detected in an assay that uses a probe. The analyte of an HSP is a ligand that interacts specifically with a binding partner member attached to a HSP arm sequence. That is, the analyte and its binding partner are a specific binding pair. An analyte may be any compound or macromolecular structure to be detected so long as some portion of it interacts specifically with the binding pair member attached to the HSP arm.

The terms “specific binding pair” and “binding pair” are used interchangeably herein to mean any pair of moieties that form a stable specific attachment to each other, by any of a variety of noncovalent interactions (e.g., hydrogen bonds, ionic bonds or interactions, hydrophobic interactions, or van der Waals forces). A member of a binding pair may be made up of any known molecular structure, including proteins, peptides, lipids, fatty acids, polysaccharides, lipopolysaccharides, nucleic acids, compounds made up of combinations of such molecular structures or analogs thereof, or an organic compound that binds specifically to another molecular structure. A specific binding pair are moieties that interact specifically, but individual members of a specific binding pair may interact specifically with other compounds, e.g., both avidin and streptavidin are ligands for biotin. The moieties of a binding pair may be of the similar or dissimilar chemical composition or structure (e.g., complementary DNA strands are considered similar chemical moieties, whereas protein-lipid interactions are considered dissimilar chemical moieties). Examples of specific binding pairs are well known in the art, such as, e.g., antibodies and antigens, haptens, or ligands, receptors or binding partners of hormones, drugs, metabolites, vitamins, and coenzymes, enzymes and their substrates, complementary nucleic acids, proteins that bind specifically to nucleic acids, chelating agents for metals, and the like. Members of a “binding pair” that are referred to herein as chemical or biochemical compounds are meant to encompass small and large (macromolecular) chemical, biochemical, and biological molecular compositions, whether made synthetically or isolated from natural sources. Generally, one member of a specific binding pair is referred to as an analyte, target, ligand, or compound of interest to be detected, and the other member of the binding pair may be referred to as a ligand or binding pair member. Those skilled in the art will appreciate that a large number of analytes may be detected using the HSP compositions and HSP-based methods described herein by choosing an appropriate binding pair member for the HSP, i.e., the invention is not dependent on any particular type or combination of binding pair members. Any target analyte and its specific binding partner may be detected using the HSP-based methods described herein so long as the binding pair interaction results in a conformational change in the HSP. One skilled in the art will further appreciate that the target analyte and its specific binding partner need not be known chemical or biochemical compounds or structures. For example, the HSP compositions and methods described herein may be used to detect new ligands for a known binding partner member, such as to detect binding of a new synthetic ligand to a known compound that is the binding pair member attached to the HSP.

By “linker element” or “linker” is meant a chain of atoms that covalently join two other functional elements of a HSP. A linker element may be any known chemical structure that joins two HSP sequences, such as, e.g., another nucleic acid sequence, abasic nucleic acid residue(s), PNA, chemical compound, or polymer such as polyethylene glycol (PEG), which may include other structures such as side-chain branches or cyclic groups.

By “sufficiently complementary” is meant a contiguous nucleic acid base sequence that is capable forming a stable hybridization duplex with another base sequence by standard hydrogen bonding between complementary bases (often referred to a base pairing, e.g., G-C, A-T or A-U pairing), under appropriate hybridization conditions. Sufficiently complementary sequences may be completely or partially complementary sequence and may contain one or more positions lacking a base (i.e., abasic residues). Contiguous bases are preferably at least about 80%, more preferably at least about 90%, and most preferably 100% complementary to the sequence to which it hybridizes.

By “hybridization conditions” is meant the cumulative biochemical and physical conditions of a reaction mixture in which complementary nucleic acid sequences bind by standard base pairing. These include, for example, solution components and concentrations, such as buffering agents, salts, detergents and the like, incubation time, temperature, and physical parameters of a reaction vessel. Appropriate hybridization conditions are well known to those skilled in the art, can be predicted based on sequence composition, or can be determined empirically by using routine testing (e.g., Sambrook et al., Molecular Cloning, A Laboratory Manual, 2^{nd} ed. (Cold Spring Harbor Laboratory Press, Cold Spring Harbor, N.Y., 1989) at §§ 1.90-1.91, 7.37-7.57, 9.47-9.51 and 11.47-11.57, particularly §§ 9.50-9.51, 11.12-11.13, 11.45-11.47 and 11.55-11.57).

HSP probes may be used in appropriate hybridization conditions that occur exclusively in solution phase (e.g., in an aqueous or organic liquid mixture) or may be “immobilized” on a support, such as a solid or gel component. In some embodiments, an immobilized probe is preferred because it facilitates separation of a bound target analyte from unbound material in a sample and/or concentrates the probe and bound analyte at a particular position of an assay device. Any known support may be used, such as matrices and particles, e.g., made of nitrocellulose, nylon, glass, polyacrylate, polystyrene, silane polypropylene, mixed polymers, or metal, such as magnetically attractable particles. Preferred supports are monodisperse magnetic spheres (e.g., uniform size±5%) to which one or more immobilized HSP is joined directly (e.g., via a direct covalent linkage, chelation, or ionic interaction), or indirectly (e.g., via one or more linkers), where the linkage or interaction joins all or a portion of the HSP to the support and is stable during the assay conditions. A mixture of supports with attached HSPs may be used, e.g., a mixture of different sizes of supports, each size being associated with a particular HSP. Other preferred supports are substantially two-dimensional surfaces that include a matrix of addressable detection loci (which may be referred to pads, addresses, or micro-locations) in an “array.” A preferred HSP array includes at least two HSPs in different locations on a support. The size and composition of a HSP array will depend on the desired end use of the array, but generally an array contains from about two to many thousands of different immobilized HSPs at different addresses, which can be made by any of a variety of known techniques, e.g., depositing or synthesizing each HSP at a predetermined location. HSPs in an array range from about 2 to about 10,000 different HSPs per support, preferably about 5 to about 1000 different HSPs per support, and more preferably about 10 to about 100 different HSPs per support.

By “label” is meant a molecular moiety or compound that can be detected or can lead to a detectable response or signal. A label may be part of the nucleic acid of a HSP or may be a separate moiety joined directly or indirectly to the HSP. A label that is part of the HSP nucleic acid includes a HSP sequence that binds to a separate nucleic acid probe. For example, if the separate probe is a molecular beacon or molecular torch, the separate probe is in the closed state that inhibits signal production when it is not bound to the HSP sequence due to the conformational state of the HSP, but when HSP switches to a different conformational state, the separate probe binds to the HSP and produces a detectable signal resulting from the separate probe's open state. In another example, a label that is part of the HSP sequence is a sequence that serves as a primer or template in a nucleic acid amplification reaction only when the HSP is in a particular conformation, and the amplified nucleic acid products are detected to indicate the conformational state of the HSP. Direct labeling of a separate moiety uses bonds or interactions that link the separate label moiety to the HSP, including covalent bonds or non-covalent interactions (e.g., hydrogen bonds, hydrophobic and ionic interactions, chelates, or coordination complexes). Indirect labeling uses a bridging moiety or “linker” which is either directly or indirectly linked to the label moiety that is joined to the HSP. Labels may be any known detectable moiety, e.g., radionuclide, ligand, enzyme, enzyme substrate, reactive group, chromophore, particle, luminescent compound (e.g. bioluminescent, phosphorescent or chemiluminescent labels), or fluorophore. Preferred labels are detectable in a homogeneous assay system, in which bound label in a mixture exhibits a detectable change compared to unbound label in the mixture, such as stability, differential degradation, or emission characteristics. Preferred labels for use in homogenous assays include known chemiluminescent compounds (e.g., U.S. Pat. Nos. 5,656,207, 5,658,737, 5,283,174, and 5,639,604). Preferred chemiluminescent labels are acridinium ester (AE) compounds, which include standard AE or derivatives thereof, e.g., naphthyl-AE, ortho-AE, 1- or 3-methyl-AE, 2,7-dimethyl-AE, 4,5-dimethyl-AE, ortho-dibromo-AE, ortho-dimethyl-AE, rheta-dimethyl-AE, ortho-methoxy-AE, ortho-methoxy(cinnamyl)-AE, ortho-methyl-AE, ortho-fluoro-AE, 1- or 3-methyl-ortho-fluoro-AE, 1- or 3-methyl-meta-difluoro-AE, and 2-methyl-AE. Methods for synthesis and attachment of labels to nucleic acids and detecting signals from labels are well known (e.g., Sambrook et al., Molecular Cloning, A Laboratory Manual, 2nd ed. (Cold Spring Harbor Laboratory Press, Cold Spring Harbor, N.Y., 1989), Chapter 10; U.S. Pat. Nos. 5,658,737, 5,656,207; U.S. Pat. No. 5,547,842, Hogan et al., U.S. Pat. No. 5,283,174, Arnold et al., and U.S. Pat. No. 4,581,333, Kourilsky et al.,).

A “homogeneous detectable label” refers to a label whose presence can be detected in a homogeneous fashion based on its physical state (e.g., in a hybridized duplex of the HSP), without physically separating the hybridized from unhybridized forms of the label in a mixture. Homogeneous detectable label systems have been described in detail (e.g., U.S. Pat. Nos. 5,283,174, 5,656,207, and 5,658,737) and preferred embodiments use labels and conditions of a homogeneous protection assay (“HPA“; see U.S. Pat. Nos. 5,283,174 and 5,639,604, Armold et al.).

By “consisting essentially of” is meant that additional component(s), composition(s) or method step(s) that do not materially change the basic and novel characteristics of a HSP or its use in detecting the presence of a target analyte may be included in the compositions, kits, or methods of the invention. Such characteristics include the ability to detect an analyte by forming a specific binding pair made up of the HSP-linked binding pair member and its ligand, the target analyte, that affects the conformational structure of the HSP and results in a positive signal or loss of a signal to indicate the presence of the analyte in the specific binding pair attached to the HSP, thus indicating the presence of the analyte in the sample. Such characteristics include at least a 10-fold increased sensitivity of detection for an analyte in a HSP-based assay compared to a radioimmunoassay (RIA) for the same analyte. Any component(s), composition(s), or method step(s) that have a material effect on the basic and novel characteristics of the present invention would fall outside of this term.

Unless defined otherwise, all scientific and technical terms used herein have the same meaning as commonly understood by those skilled in the relevant art. General definitions of many of the terms used herein are provided, for example, in Dictionary of Microbiology and Molecular Biology, 2nd ed. (Singleton et al., 1994, John Wiley & Sons, New York, N.Y.) or The Harper Collins Dictionary of Biology (Hale & Marham, 1991, Harper Perennial, New York, N.Y.). Unless mentioned otherwise, the techniques employed or contemplated herein are standard methodologies well known to one of ordinary skill in the art. The examples included herein illustrate some embodiments of the invention.

A hybridization switch probe (HSP) is a nucleic acid composition that includes the following functional elements: a first arm sequence that has an attached binding partner member, a second arm sequence that has an attached label, and a support sequence that is complementary to an arm sequence. Structural elements of a HSP may perform one or more functions of the HSP. For example, in one embodiment, the first or second arm sequence may also be the support sequence (i.e., the arm sequences are complementary to each other). In another embodiment, the arm sequences are independent sequences from the support sequence (i.e., three oligomer sequences, of which two are arm sequences and one is the support sequence. The arm and support sequence elements may be on separate oligomers that become linked to each other by non-covalent binding, such as complementary base pairing under hybridization conditions, or the arm and support sequences may be covalently linked, directly or indirectly, in preferred embodiments. Any known method may be used to link these nucleic acid sequences, including nucleotide and non-nucleotide linker elements. In a preferred embodiment, the arm sequence and support sequence are linked by a short nucleic acid sequence that is not substantially complementary to the sequences of the arm or support elements, preferably of about 5 to 15 residues in length. For example, an arm sequence may be linked to a support sequence by a short homopolymeric sequence, such as a poly-A or poly-T sequence. Other examples of linker elements include abasic nucleic acid residues, peptide nucleic acids (PNA), or other polymers, such as, e.g., polyethylene glycol (PEG), polysaccharides, or polypeptides.

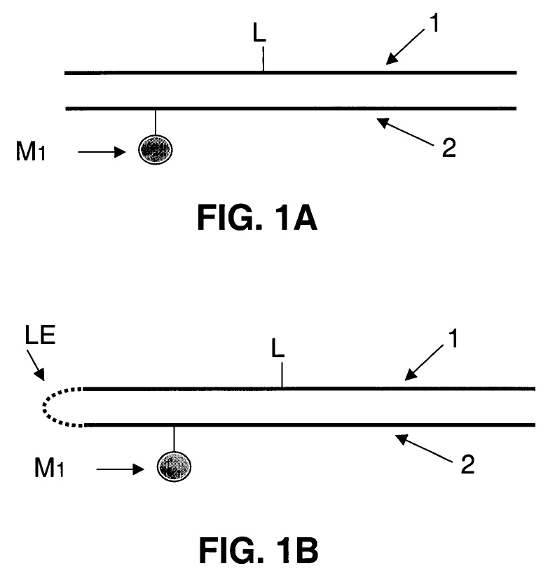

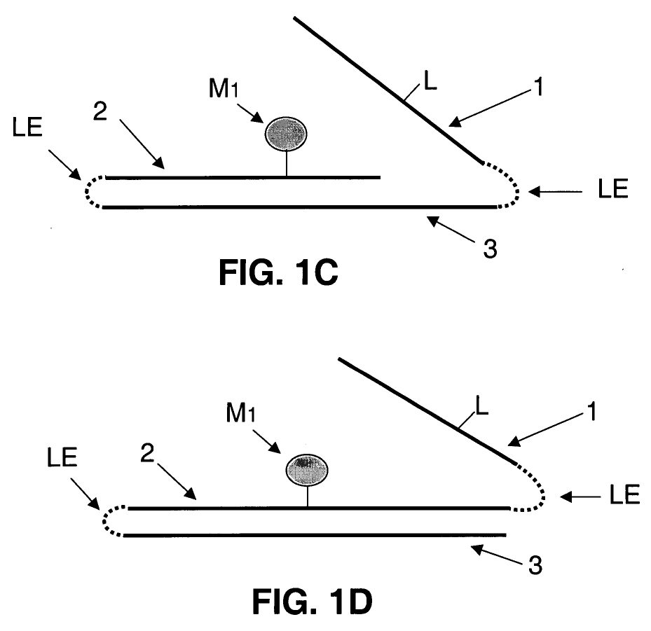

FIGS. 1A to 1D illustrate some hybridization switch probe (HSP) embodiments. Referring to FIG. 1A, one HSP embodiment is made of two complementary nucleic acid sequences that are in separate strands, which under hybridization conditions, join by standard base pairing to form a duplex. The embodiment illustrated in FIG. 1A shows a first strand (1) with an attached label (L) and a second strand (2) with an attached member of a binding pair (M₁) which is specific for the analyte to be detected. Those skilled in the art will appreciate that the positions of the label and binding pair member may be reversed relative to the strands of the HSP. FIG. 1A illustrates a two-strand embodiment in which one arm sequence (e.g., 1) is functionally the support sequence for the other arm sequence (e.g., 2).

Referring to FIG. 1B, the HSP embodiment illustrated is similar to that of FIG. 1A, but is made up of two complementary nucleic acid sequences (1, 2) that are covalently joined by a linker element (LE). In this embodiment, one arm sequence functions as the support sequence for the other arm sequence.

Referring to FIG. 1C, this HSP embodiment is made of three nucleic acid sequences (1, 2, 3) that are covalently joined by linker elements (LE), where the two arm sequences (1 and 2) flank a separate support sequence (3). In the illustrated embodiment, the first arm sequence (1) has an attached label (L) and the second arm sequence (2) has an attached member of a binding pair (M₁), but the positions of the label and binding pair member may be reversed relative to the arm sequences in another embodiment (i.e., 1 attached to the binding pair member and 2 attached to the label). The intervening support sequence (3) is at least partially complementary to both arm sequences (1 and 2), so that each arm sequence under appropriate hybridization conditions can form a duplex with the support sequence (as shown by the hybridization duplex of sequences 2 and 3).

Referring to FIG. 1D, the HSP embodiment is made of three nucleic acid sequences (1, 2, 3) that are covalently joined by linker elements (LE) but in a different order than the embodiment illustrated in FIG. 1C. In FIG. 1D, the first arm sequence (1) with an attached label (L) is joined by a linker element (LE) to the second arm sequence (2) with an attached binding pair member (M₁), which is joined by another linker element (LE) to the support sequence (3), which is at least partially complementary to both arm sequences (1 and 2). Either arm sequence 1 or 2 can form a hybridization duplex with the support sequence under appropriate hybridization conditions, as illustrated in FIG. 1D by the duplex formed between sequences 2 and 3.

Although many embodiments of a functional HSP are envisioned, preferred embodiments are those that covalently link the arm and support sequence elements, as illustrated in FIGS. 1B, 1C and 1D. Such structures utilize the kinetic advantages of intramolecular hybridization to join the complementary arm and support sequences, resulting in the conformational changes that are used to assay for an analyte specific for the binding pair member attached to the HSP.

An embodiment of a HSP-based assay for an analyte is illustrated in FIG. 2. The illustrated HSP, similar to that of FIG. 1D, includes a label on the first arm sequence that is an acridinium ester (AE) compound that emits a chemiluminescent signal. In the upper portion of FIG. 2, the HSP is in a first conformation in which the arm sequence (2) attached to the binding pair member (M₁) specific for the target analyte (M₂) is in a hybridization duplex with the support sequence (3) because the analyte is not present. The duplex of sequences 2 and 3 limits formation of a duplex between sequences 3 and 1 because a portion of the support sequence that is complementary to arm sequence 2 overlaps with a portion of the support sequence that is complementary to arm sequence 1. When the analyte (M₂) is present in the assay mixture, the analyte and the binding pair member (M₁) form a specific binding pair complex (BPC) that destabilizes the duplex of sequences 2 and 3, allowing sequences 3 and 1 to form a stable duplex, illustrated in the lower portion of FIG. 2. In this second conformation, the AE label is protected from hydrolysis by the hybridization duplex of sequences 1 and 3 and the chemiluminescent signal from the AE label can be detected by using a hybridization protection assay (HPA) format (U.S. Pat. Nos. 5,283,174 and 5,639,604). Briefly, the AE label present on a single-stranded sequence is selectively degraded, such as by using an acidic (e.g., pH 5 to 6), or a basic (e.g., pH 8 to 9) solution, or an oxidizing agent, while AE present on a strand in a double-stranded structure is protected from degradation. Then the undegraded AE label is activated (e.g., by treating with H₂O₂) to produce a chemiluminescent signal that is detected by standard methods (e.g., luminometry). The detected signal in this embodiment is proportional to the amount of analyte present in the assayed sample.

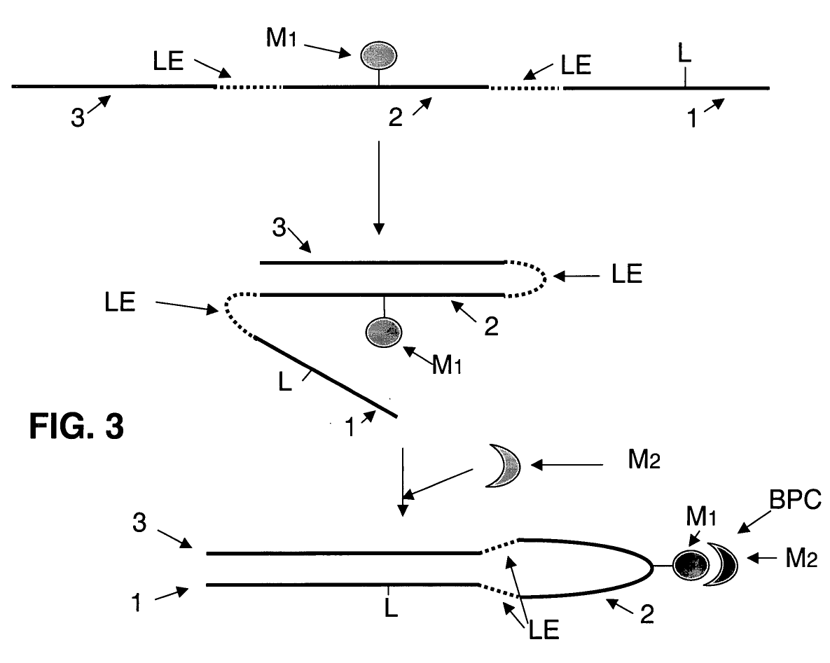

Another assay embodiment, illustrated in FIG. 3, uses a HSP in which the first arm sequence (1), the second arm sequence (2), and the support sequence (3) are joined in that order by a linker elements (LE), as shown in the upper portion. The middle portion of FIG. 3 shows this HSP in a first conformation in the absence of analyte, similar to that of FIG. 1 D, in which sequences 2 and 3 are in a hybridization duplex, leaving the labeled arm sequence substantially single-stranded. As shown in the bottom portion of FIG. 3, when the analyte (M₂) is present and binds to the binding partner member (M₁), the binding partner complex (BPC) forms, destabilizing the duplex of sequences 2 and 3. This permits sequences 1 and 3 to form a hybridization duplex and arm sequence 2 loops out with the attached BPC. If the label is an AE compound, the HPA detection format is followed and the detected chemiluminescent signal is proportional to the amount of analyte present in the sample.

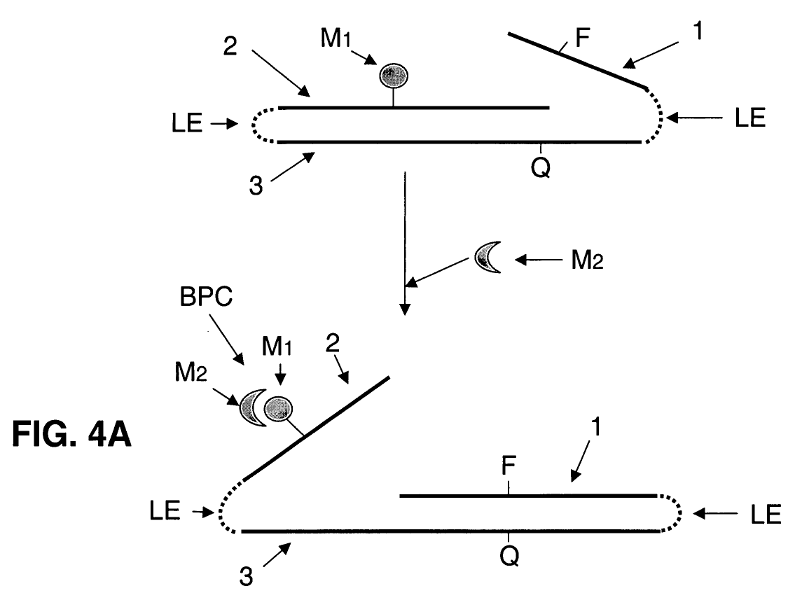

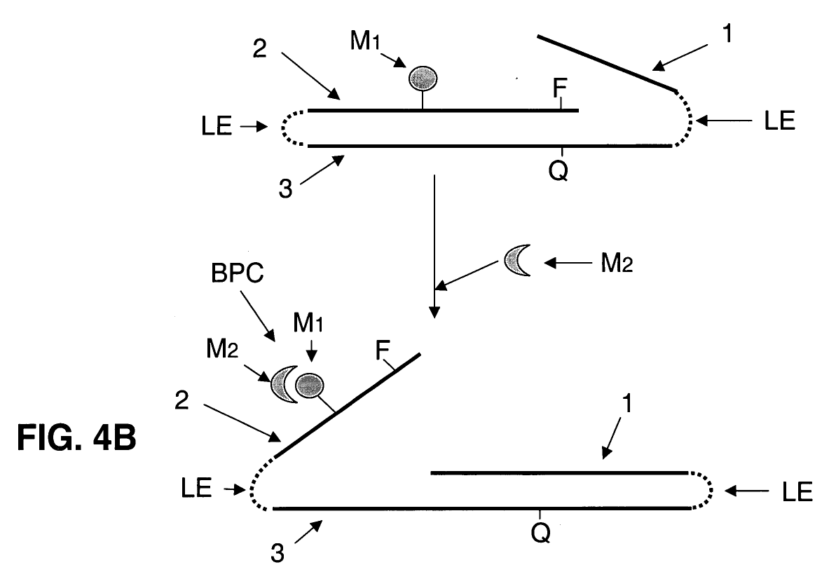

Another assay embodiment, illustrated in FIG. 4A, uses a HSP with a fluorophore label. The HSP is similar to that of FIG. 1C, but the first arm sequence (1) is labeled with a fluorophore (F) and the support sequence (3) has an attached quencher compound (Q) that inhibits fluorescent emission when the fluorophore and quencher are in close proximity. The first arm sequence (1) is joined by a linker element (LE) to the support sequence (3) which is joined by a linker element (LE) to the second arm sequence (2) with its attached binding pair member (M₁) which is specific for the target analyte (M₂). As shown in the upper portion of FIG. 4A, in the absence of analyte, the HSP is in a first conformation in which sequences 2 and 3 are hybridized to form a duplex, leaving sequence 1 substantially single-stranded and free to move so that the attached fluorophore is distant from the quencher, resulting in a detectable fluorescent signal. When the analyte is present in the assayed sample, the analyte (M₂) attaches to the binding pair member (M₁) on the second arm sequence (2) and the resulting binding pair complex (BPC) that destabilizes the duplex made up of sequences 2 and 3, which permits sequences 1 and 3 to hybridize forming the second conformation shown in the lower portion of FIG. 4A. The second conformation with the duplex made up of sequences 1 and 3 brings the fluorophore and quencher into close proximity, which decreases the amount of detectable fluorescent signal, and thus that the amount of analyte is inversely proportional to the detectable signal. That is, the amount of analyte present in the sample is proportional to the inhibition of signal resulting from the second conformation relative to a control mixture that does not contain analyte and produces a signal resulting from the first conformation.

Those skilled in the art will appreciate that the positions of the fluorophore and quencher compound may be varied on HSP sequences to achieve substantially the same result as illustrated in FIG. 4A, so long as the conformation when the analyte is present places the fluorophore and quencher compound in close proximity to decrease fluorescence. For example, the fluorophore may be attached to the support sequence, the quencher compound to the first arm sequence, and the binding pair member to the second arm sequence to achieve substantially the same result as the embodiment illustrated in FIG. 4A. That is, when the analyte is absent the HSP is in a first conformation in which the support sequence 3 with the attached quencher compound binds to arm sequence 2 with the attached binding pair member (M₁), thereby separating the fluorophore and the quencher compound. In this conformation, because the fluorophore and quencher compound are relatively distant, the HSP label emits fluorescence. In contrast, when the analyte (M₂) is present the HSP switches to its second conformation because the analyte binds to its binding pair member to form a specific binding pair complex (BPC) that destabilizes the duplex of sequences 2 and 3, favoring the formation of a hybridization duplex made up of sequences 1 and 3, which brings the fluorophore and quencher compound into close proximity, thereby limiting fluorescence from the HSP. Thus, like the embodiment illustrated in FIG. 4A, this embodiment decreases fluorescence when the analyte is present in the sample compared to a control or sample that contains no analyte. Preferred embodiments of such HSP-based assays provide a fluorescent signal that is inversely proportional to the amount of analyte in the sample.

FIG. 4B illustrates an embodiment of a HSP-based assay that provides a positive signal when the analyte is present in the sample. In this embodiment, the both the fluorophore label (F) and the binding pair member (M₁) are present on arm sequence 2 and the quencher compound (Q) is on support sequence 3. In the absence of analyte, as shown in the upper portion, the first conformation of the HSP is favored in which sequences 2 and 3 form a duplex that brings the fluorophore and quencher compound into close proximity, thus limiting fluorescence. When the analyte (M₂) is present, as shown in the lower portion, the analyte and its binding pair member (M₁) form a specific binding pair complex (BPC) on arm sequence 2, which destabilizes the duplex of strands 2 and 3 converting the HSP to its second conformation in which a duplex of strands 1 and 3 is favored. The second conformation effectively separates F and Q so that F emits fluorescence. Thus, in the embodiment shown in FIG. 4B, when the analyte is present a positive signal is produced that is proportional to the amount of analyte in the sample.

The methods that use a HSP embodiment that includes two arm-sequences present in a single molecular structure use the advantages of intramolecular hybridization to efficiently form duplexes involving the support sequence and one of the arm sequences in the same hybridization conditions. A change in the stability of a duplex that involves one of the arm sequences and the support sequence is counterbalanced by a change in the stability of a duplex made up of the other arm sequence and the support sequence. That is, a condition that destabilizes the first hybridization duplex favors the formation of the second hybridization duplex, resulting in a shift in the same HSP from a first conformation to a second conformation. For example, if a duplex made up of the first arm and the support sequences is destabilized by formation of a specific binding pair complex that includes the analyte and its binding partner, then formation of another duplex made of the second arm and the support sequences is favored.

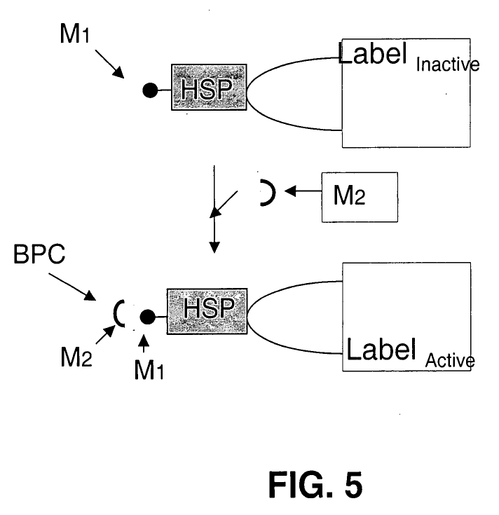

FIG. 5 illustrates this in a generic HSP and HSP-based assay. In the upper portion, in the absence of analyte, the HSP with its attached binding pair member (M₁) is in a first conformation in which the label attached to the HSP is in an inactive state. In the lower portion, in the presence of analyte, the binding pair member (M₁) joins with the analyte (M₂) to form a binding pair complex (BPC) that shifts the HSP to a second conformation in which the label is in an active state. Thus, the presence of the analyte in the sample is detected by measuring a signal from the label that results from a shift from a first to a second conformational state of the HSP.

Although not wishing to be bound by any particular mechanism or interpretation, the different conformational states of HSP are generally thought to result from the relative stability of a nucleic acid duplex structure in the presence or absence of a specific binding pair complex attached to the HSP. In the absence of the target analyte, the HSP structure favors formation of a relatively stable hybridization duplex formed between the support sequence and the sequence with an attached binding pair member, whereas in the presence of the target analyte a binding pair complex forms that destablizes this hybridization duplex, probably due to a steric effect. Destabilization of the first hybridization duplex results in a relatively stable hybridization duplex formed between the support sequence and another sequence that does not have the attached binding pair complex. The binding pair member that forms the specific binding pair complex with the analyte may be any ligand combination sufficient to produce the conformational shift from one state to another in the HSP and the detectable signal resulting from this conformational shift indicates the presence of the analyte in the sample.

From the illustrations and descriptions of various embodiments of HSP and HSP-based assays provided herein, those skilled in the art will appreciate that many different forms of HSP may be used to detect analytes. For example, an HSP-based assay may use a HSP that forms a first conformation by intermolecular hybridization, as illustrated in FIG. 1A, or a HSP that relies on intramolecular hybridization to determine its conformational states, as illustrated in FIGS. 1B to 1D. Embodiments that use an AE label would have the label protected by the duplex conformation as shown in FIGS. 1A and 1B, but when the analyte for the binding pair member (M₁) attaches and forms a specific binding pair complex, the duplex conformation would be destabilized allowing the AE label to be degraded in a hybridization protection assay format. Thus, in the absence of analyte, the AE label is protected from hydrolysis and a positive chemiluminescent signal is detected, but when analyte is present in the assay, the duplex would is destabilized and the AE label would become susceptible to hydrolysis, resulting in decreased chemiluminescence. In other embodiments, such as those illustrated in FIGS. 4A and 4B, the label may be a fluorophore that emits fluorescence when the HSP is in one conformational state and decreases fluorescent emission when the HSP shifts to another conformation state that results from formation of a binding pair complex that includes the analyte. Although FIGS. 4A and 4B show a fluorophore label associated with a quencher compound that modulates the fluorescence emission depending on the proximity of the fluorophore to the quencher compound, those skilled in the art will appreciate that other forms of fluorescence signal generation may be used. For example, a HSP may be labeled by using a combination of fluorescence resonance energy transfer (FRET) dyes to achieve a measurable change in fluorescence dependent on the conformational state of the HSP. In a HSP-based assay that uses FRET, a fluorescent donor molecule transfers energy via a dipole-dipole interaction to an acceptor fluorophore that is in close proximity (e.g., 10-70 ÅA), whereby the donor's fluorescence is reduced and the acceptor's fluorescence is increased, so that the detected signal change indicates the HSP conformational change due to analyte binding. In another example, a fluorophore labeled HSP may be used to make fluorescence polarization measurements to provide information on the HSP conformational state in an assay, preferably to provide a quantitative measurement of fluorescence polarization that indicates the quantity of analyte in the tested sample. Fluorescent compounds are well known, including fluorescein dyes (e.g., FITC, 5-carboxy fluorescein, 6-carboxy fluorescein, fluorescein diacetate, naphthofluorescein, HEX, TET, 5-carboxy JOE, 6-carboxy JOE, Oregon Green 488, Oregon Green 500, Oregon Green 514, erythrosin, eosin), rhodamine dyes (e.g., rhodamine green, rhodamine red, tetraethylrhodamine, 5-carboxy rhodamine 6G (R6G), 6-carboxy R6G, tetramethylrhodamine (TMR), 5-carboxy TMR or 5-TAMRA, 6-carboxy TMR or 6-TAMRA, rhodamine B, X-rhodamine (ROX), 5-carboxy ROX, 6-carboxy ROX, lissamine rhodamine B, Texas Red), BODIPY dyes, cyanine dyes (e.g., Cy3, Cy3.5, Cy5, Cy5.5, Cy7), phthalocyanine dyes, coumarin dyes (e.g.,7-hydroxycoumarin, 7-dimethylaminocoumarin, 7-methoxycoumarin, 7-amino-4-methylcoumarin-3-acetic acid (AMCA)), pyrene or sulfonated pyrene dyes, phycobiliprotein dyes (e.g., B-phycoerythrin (B-PE), R-phycoerythrin (R-PE), and allophycocyanin (APC)), squariane dyes, Alexa dyes (Alexa 350, Alexa 430, Alexa 488, Alexa 532, Alexa 546, Alexa 568, Alexa 594), Lucifer yellow and zanthene. Another example of a label to produce a detectable signal change dependent on HSP conformational states is a dye that selectively intercalates in DNA in one conformation state (e.g., double-stranded) compared to another conformational state (e.g., single-stranded), or a dye that has a different absorption and emission wavelengths characteristic of the dye bound to double-stranded or single-stranded DNA, which is particularly useful for a HSP that uses intermolecular hybridization (e.g., see FIG. 1A) to shift between double- and single-stranded conformations dependent on formation of a binding pair complex that includes the analyte. Such dyes are well known, e.g., acridine orange, acridine red, toluidine blue (2-amino-7-dimethylamino-3-methylphenothiazinium chloride), thiazole orange, propidium iodide (3,8-Diamino-5-(3-diethylaminopropyl)-6-phenyl-phenanthridinium iodide methiodide), hexidium iodide, dihydroethidium, ethidium bromide, ethidium monoazide, Sybr green, Sybr gold, cyanine dyes (e.g., SYTO™, TOTO™, YOYO™ and BOBO™ dyes, Molecular Probes, Eugene, Oreg.), and the like. Another example of a label that may be used to produce a detectable signal change dependent on HSP conformational states is a chromophore that produces a detectable signal that relies on one conformational state of the HSP. For example, colloidal particles (e.g., colloidal gold or silver) or nanoparticles with attached oligonucleotide sequences may form a detectable structure (e.g., cluster, aggregated, or crystaline structures) associated with the HSP when it is in one conformational state. Other chromophores may be used to produce a color change dependent on the conformational state of the HSP, such as resulting from chromophores on different HSP portions being brought into proximity to each other to produce the color change specific for one HSP conformational state. Another example of a label that produces a colorimetric signal is a moiety that participates in a reductive-oxidative (RedOx) reaction that occurs when the HSP is in a particular conformation, i.e., atoms change their oxidation state in response to a HSP conformational change mediated by the presence of the analyte, thereby signaling the presence of the analyte. Another example of a label that results in a detectable signal change dependent on the HSP conformational state is a moiety whose binding results in an electronic signal, such as formation of gold-dithiol nano-networks with non-metallic electronic properties where such a network forms preferentially with one HSP conformational state.

In other embodiments, a portion of the HSP nucleic acid may serve as a component in the detection step. In one example, a portion of the HSP nucleic acid may serve as the label that is detected to indicate the HSP conformational state dependent on the analyte binding to the binding pair member. That is, in one conformational state, a portion of the HSP may form a structure or associate with other nucleic acids to for a structure that provides a detectable signal, such as a branched, multi-arm, knotted, circular, or catenated DNA structure that is detected. Thus, the conformational state of the HSP, alone or in association with other components, indicates the presence of the analyte.

In other embodiments, a portion of the HSP may serve as a component in a nucleic acid amplification step that leads to a detectable signal. An unhybridized arm sequence of the HSP may serve as a primer or substrate for nucleic acid amplification by using any well-known method, e.g., polymerase chain reaction (PCR) or a transcription associated amplification. For example, referring to FIG. 2, a portion of the free first arm sequence (1) of the conformation shown in the upper portion or a portion of the free second arm sequence (2) of the conformation shown in the lower portion may serve as a primer for amplification of a nucleic acid that is partially complementary to the free arm sequence. Alternatively, a sequence in the free first arm sequence (1) of the upper portion of FIG. 2 or of the free second arm sequence (2) of the lower portion of FIG. 2 may serve as a substrate for primer(s) used in a nucleic acid amplification reaction so that at least a portion of the free arm sequence is amplified. Such amplified sequences may be used in a detection step that couples the advantages of nucleic acid amplification (i.e., producing many copies to amplify a detectable moiety) to the HSP conformational change used to detect an analyte. Those skilled in the art will appreciate than any HSP format or conformation that includes an unhybridized sequence may be coupled to a nucleic acid amplification reaction that uses all or a portion of the unhybridized HSP sequence. For example, referring to FIG. 5, the “label” may be a portion of the HSP that participates as a primer or substrate in a nucleic acid amplification reaction when the HSP is in a particular conformation. In the upper portion of FIG. 5, in the absence of the analyte, the HSP is in an inactive conformation that does not serve as a primer or template for a nucleic acid amplification, thus producing little or no detectable signal associated with an amplified nucleic acid sequence. In the lower portion of FIG. 5, in the presence of the analyte, the HSP is in an active conformation that participates as a primer or template in a nucleic acid amplification reaction, resulting in amplified nucleic acid sequences that provide a detectable signal to indicate the presence of the analyte in the tested sample. By coupling a HSP-based assay to a nucleic acid amplification step, the sensitivity of the HSP-based assay may be increased because of the signal amplification achieved by using nucleic acid amplification.

Other embodiments of HSP-based assays may include signal amplification that relies on cycling probe moieties that bind to a portion of the HSP in one conformation. Again referring to FIG. 5, in this embodiment the “label” is a portion of the HSP that binds to a second nucleic acid probe that only produces a signal when it is bound to the HSP label portion. In the upper portion of FIG. 5, in the absence of the analyte, the HSP is in an inactive conformation that does not permit binding of the second probe, thereby preventing signal emission from the second probe in the mixture. In the lower portion of FIG. 5, in the presence of the analyte, the HSP is in an active conformation that binds the second probe, thereby permitting signal emission from the second probe to indicate the conformational change in the HSP resulting from the analyte present in the sample. Then, the second probe bound to the active conformation of the HSP, is physically disrupted (e.g., cleaved), to separate the second probe from the HSP and prevent reformation of the inhibited form of the second probe. That is, the disrupted second probe continues to emit signal even when not bound to the HSP. The active HSP, meanwhile, may bind another second probe which is then disrupted, producing a series of second probes that produce detectable signals. Each active HSP conformation is able to bind multiple copies of the second probe, thereby amplifying the signal emitted from the second probe. Disruption of the bound second probes may be accomplished by using enzymatic means, such as, e.g., by RNase H digestion of an RNase H-sensitive scissile link in the second probe that is only recognized by the enzyme when the probe is bound to the HSP. In another example, disruption of the bound second probes may be performed by a restriction endonuclease that only cleaves the recognition sequence in the probe when the probe is bound to the HSP.

A HSP may use any binding pair member that forms a specific binding pair complex with the analyte to be detected. Analytes include, but are not limited to, membranes or membrane fragments (e.g., cellular, nuclear or organelle), receptors (e.g., for a cytokine, hormone, opioid, steroid or infectious agent), cells, bacteria, viruses, prions, toxins, proteins, carbohydrates, lipids, enzymes, proteases, kinases, antigens, antibodies or antibody fragments, lectins, nucleic acids, and any biological, organic or organo-metallic species that can interact with a ligand or reactive substrate. Combinations of specific binding pair members are well known in the art and any binding pair member that can be associated with the HSP by using well known attachment methods while retaining its ability to bind its ligand may be used. In some embodiments, a portion of the nucleic acid structure of the HSP serves as the binding pair member for the analyte to be detected. In a preferred embodiment, a portion of the HSP may be an aptamer that specifically binds the analyte which results in a conformational change in the HSP that is detected by a signal change from the HSP label as described above.

Similarly, those skilled in the art will understand that the methods illustrated in the figures are only some embodiments of the detection assays that make use of HSPs and that other known assay formats are encompassed by the invention, e.g., competitive assays. For example, an HSP-based assay may include a HSP-binding pair member that is specific for both the analyte of interest and for another ligand, such that the analyte and additional ligand compete for binding to the HSP-binding pair member and the different binding pair complexes mediate different conformational changes in the HSP which can be detected as a signal change that indicates the presence or relative amount of analyte in the sample. In one such embodiment, the binding pair complex made up of the analyte and the HSP-bound binding pair member mediates a HSP conformational change that results in a positive signal, whereas the binding pair complex made up of the other ligand and the binding pair member results in a HSP conformation that emits no detectable signal, such that competition between the analyte and the ligand results in an increased signal proportional to the amount of analyte in the sample. In another example of a competition assay format, the assay includes a HSP-bound binding pair member for the analyte and a known amount of free binding pair member for the same analyte, such that the two binding pair member forms compete for binding to the analyte, i.e., binding of one excludes binding of the other to the same analyte molecule. In this embodiment, a HSP conformational change mediated by formation of a binding pair complex on the HSP, which produces a signal change, occurs when the sample contains a sufficient amount of the analyte to bind to both the free binding pair members and the HSP-bound binding pair members. In preferred embodiments of HSP-based competition assays, the signal resulting from analyte bound to the HSP-binding pair member is proportional to the amount of analyte in the tested sample, i.e., the assay provides quantitative results.

Similarly, an assay may use one or more HSPs in solution phase, or one or more HSPs bound to a support, each HSP specific for a particular analyte. Supports for such assays may include particles, matrices, or solid supports to which one or more HSPs are attached, such as in an array format, which are contacted with one or more samples when the assay is performed. Solution phase assays are advantageous because of the kinetic advantages of binding reactions that occur in solution compared to immobilized components. Support bound assays are advantageous because of their ability to concentrate analytes from a relatively dilute sample into a limited space or position for detection and because they may be used for high through-put testing, e.g., on an array. Detection of signals emitted from conformational state changes of multiple different HSP in solution or bound to a support may be achieved by using any of a variety of well known methods, e.g., by use of a detector that collects positional and/or time-correlated signals at one or more wavelengths, frequencies, energy levels, or similar characteristics appropriate for the HSP label chosen, such as by using a detector positioned to collect emission data from an immobilized HSP system as a result of irradiation by the one or more excitation wavelengths directed to specific positions of an array.

The invention encompasses kits and systems that use the HSP compositions and/or HSP-based methods described herein. A kit includes at least one HSP specific for an analyte, and may include multiple different HSPs specific for the same analyte or for different analytes. Different HSPs in a kit may have substantially the same format (e.g., any of those as illustrated in the figures), and may differ only in the specific binding pair that each HSP detects. Alternatively, a kit may include HSPs of different formats (e.g., combinations of at least two embodiments illustrated in the figures) which all detect the same or different specific binding pairs. In addition to the HSP component(s) of a kit, the kit may include additional reagents used in performing an assay, such as, e.g., reagents for sample preparation before the HSP and sample are mixed, and/or reagents to obtain appropriate hybridization conditions, e.g. buffering agents, chelators, salts, or mixtures of such reagents, and/or reagents to produce a signal from the label attached to a HSP, e.g., an enzyme or substrate, a hydrolyzing agent, and the like. An instrument system that is used for performing HSP-based assays is also encompassed by this invention. Such a system may be simple including, e.g., a container or array having one or more HSPs therein, in which the detection method is conducted by manual manipulations. Such a system may be more complex including components for automated performance of the detection method steps and/or additional steps, such as those involved in sample preparation. Automated steps may include dispensing reagents, mixing the sample with a HSP reagent and/or other reagents, incubating mixtures to permit formation of a binding pair complex that leads to a HSP conformational change, and detecting a signal change resulting from binding pair complex formation and a HSP conformational change. Such systems may include signal detection instrumentation, e.g., to detect emission or absorbance of signal resulting from luminescent, fluorescent, colorimetric, electronic, or other types of signals. Preferred embodiments of systems detect a signal and provide a qualitative or quantitative output proportional to the amount of analyte present in the tested sample.

The compositions, methods, kits and systems of the invention are useful for detecting a variety of target analytes in a variety of samples, e.g., detection of a protein, carbohydrate, lipid, fatty acid, or macromolecular complex that indicates the presence of a drug, infectious agent, toxin, or the like in a biological, industrial, food, or environmental sample. Other examples of applications of the compositions and methods of the invention include diagnostic detection of antigens, antibodies, or infectious agents such as a microbe, virus, or prion in a biological sample. Because of the relative simplicity of the HSP-based methods, such assays are useful for high through-put screening of many samples to detect the presence of a ligand, such as for screening many environmental or food samples for the presence of an infectious agent or toxin. Because of the simplicity and sensitivity of the HSP-based methods, the assays are useful for rapid testing of samples outside of a laboratory, e.g., for testing environmental sites, food processing facilities, or screening an area for forensic evidence.

The examples that follow illustrate some embodiments of the invention. A model system used to illustrate HSP-based methods uses the binding pair of biotin and streptavidin. In embodiments that use a chemiluminescent label, the compositions and methods of the invention allow detection of an analyte that is a member of a specific binding pair to a level of 10⁻¹⁷ to 10⁻¹⁸ moles, which is generally 10² to 10⁵ more sensitive than detection of the same analyte in the same specific binding pair in another assay format, e.g., a typical RIA. The compositions and methods of the invention may used other labels on the HSP, e.g., a fluorescent label, which may provide a different level of assay sensitivity. The assay sensitivity for a particular analyte thus may be varied by selecting a label that achieves the desired sensitivity level for the analyte to be detected. For example, a higher level of HSP-based assay sensitivity may be needed for diagnostic detection of an infectious agent such as HIV-1 in a biological sample than would be required for detection of Escherichia coli in an environmental water sample. The HSP compositions and HSP-based detection methods provide an assay response that is almost linear over a dynamic range of about three to four logs which makes them particularly useful for applications which require quantitative results. HSP compositions may be used in a wide variety of general methods for detection of a target analyte, such as in a standard or competitive assay format to provide a positive or inhibited signal output. HSP compositions and methods may be used in a solution phase system or in a system that uses one or more immobilized components, e.g., in an array, and preferred embodiments use a homogeneous detection system. In any of these formats, HSP-based methods are simple to perform, are effective over a wide temperature range (e.g., about 20° C. to 50° C.), and require relatively simple conditions that allow nucleic acid hybridization. Thus many embodiments of HSP-based methods can be performed without requiring complicated procedures or devices as used in other methods, such as in nucleic acid amplifications. Because of the relative simplicity of HSP-based methods, HSP compositions and HSP-based assays may be readily performed manually or adapted for use in automated systems or devices.

In the examples that follow, the reagents used typically were as follows, although those skilled in the art will appreciate that a variety of known conditions that allow association of specific binding pairs and hybridization of nucleic acids may be used. Probe reagent contained one or more labeled probes in a solution made up of either: 100 mM lithium succinate, 3% (w/v) LLS, 10 mM mercaptoethanesulfonate, and 3% (w/v) polyvinylpyrrolidon, or 100 mM lithium succinate, 0.1% (w/v) LLS, and 10 mM mercaptoethanesulfonate. Hybridization reagent contained either 190 mM succinic acid, 17% (w/v) LLS, 100 mM lithium hydroxide, 3 mM EDTA, and 3 mM EGTA, at pH 5.1, or 100 mM succinic acid, 2% (w/v) LLS, 100 mM lithium hydroxide, 15 mM aldrithiol-2, 1.2 M lithium chloride, 20 mM EDTA, and 3.0% (v/v) ethanol, at pH 4.7. For probes labeled with a AE compound, Selection reagent used to initiate AE hydrolysis contains 600 mM boric acid, 182.5 mM sodium hydroxide, 1% (v/v) octoxynol (TRITON® X-100), at pH 8.5 to 9.6, and Detection reagents were Detect Reagent I, which contains 1 mM nitric acid and 32 mM hydrogen peroxide, and Detect Reagent II, which is 1.5 M sodium hydroxide. Chemiluminescence (expressed as relative light units or “RLU”) was detected using a luminometer (e.g., LEADER® HC, Gen-Probe Incorporated, San Diego, Calif.), and fluorescence was detected using a fluorometer.

Typically, a reaction that used an AE-labeled HSP involved the following steps. A reaction mixture contained a known amount of AE-labeled HSP mixed with a sample containing the analyte for the HSP in an aqueous solution under hybridization conditions (i.e., in hybridization reagent). The reaction mixture was incubated for 10-15 min at about 22-37° C. to allow formation of the binding pair complex (the analyte and its binding partner on the HSP) and the resulting conformational change in the HSP. Then, an equal volume of selection reagent was added to the reaction mixture which was mixed, covered with a layer of inert oil to reduce evaporation, incubated at 37-60° C. for 10 min to hydrolyze AE not present in a nucleic acid duplex structure, and cooled to room temperature. Chemiluminescence from the remaining unhydrolyzed label was initiated by adding Detect Reagents I and II sequentially, and the chemiluminescence was detected as relative light units (RLU) for about 0.5-2 sec by using a luminometer (HC LEADER®, Gen-Probe Incorporated), substantially as described in U.S. Pat. No. 5,658,737 at column 25, lines 27-46, and Nelson et al., 1996, Biochem. 35:8429-8438 at 8432).

A reaction that used a fluorophore-labeled HSP involved the incubation step to allow formation of the analyte-binding partner binding pair complex on the HSP resulting in the conformational change in the HSP and a detection step to detect the fluorescent signal associated with the HSP conformational change. Because the fluorophore did not require a chemical activation step, the selection step used for the AE-label described above was not included. That is, a typical reaction using a fluorophore-labeled HSP included the following steps. A reaction mixture contained a known amount of fluorophore-labeled HSP mixed with a sample containing the analyte for the HSP in an aqueous solution under hybridization conditions (i.e., in hybridization reagent) which was incubated for 10-15 min at about 22-37° C. to allow formation of the binding pair complex and the resulting HSP conformational change. Using a fluorometer, the fluorescent signal was detected and measured using standard procedures. Detection may include illuminating the reaction mixture with an excitation wavelength specific for the fluorophore label, followed by detection of the fluorescent signal at the appropriate emission wavelength or range of wavelengths to detect the fluorescent signal that indicates the HSP conformational change. Those skilled in the art will appreciate that multiple fluorescent signals from different HSPs each labeled with a different fluorophore may be detected by appropriately setting the excitation and/or emission spectra for the fluorophore labels used, e.g., by choosing different wavelengths to detect maximal or non-overlapping emissions for each fluorophore label used.

The examples that follow illustrate the principles and advantages of the invention, including its simplicity and sensitivity of detection.

### EXAMPLE 1

### Design, Synthesis and Testing of Hybridization Switch Probe Embodiments

Hybridization switch probes of the general format as illustrated in FIG. 1C were designed using one support sequence (SEQ ID NO: 8) joined to various combinations of arm sequences (SEQ ID Nos. 1 to 7) by using linker elements, each made up of a short homopolymer sequence (T₅). In each of the designed embodiments, both of the arm sequences can hybridize to a sequence in the support sequence. Oligomers containing these combined sequences were synthesized by using standard chemical reactions to make HSP oligomers having the sequences shown in Table 2 (SEQ ID Nos. 9 to 15). Table 1 shows the arm sequences used in designing the HSP oligomers, and Table 2 shows the complete HSP sequences, with the arm sequences in italics and the support sequences underlined. In the HSP oligomers, the 5′ or first arm sequences (SEQ ID Nos. 1-4) were labeled with an AE compound by using a covalent chemical linkage (between residues 6 and 7 for SEQ ID NO:1, between residues 5 and 6 for SEQ ID NO:2, between residues 7 and 8 for SEQ ID NO:3, and between residues 8 and 9 for SEQ ID NO:4), by using well known methods (e.g., U.S. Pat. No. 5,185,439, Arnold et al.). In the HSP oligomers, the 3′ or second arm sequences (SEQ ID Nos. 5 to 7) were labeled with biotin by using a covalent chemical linkage (between residues 7 and 8 for SEQ ID NO:5, and between residues 8 and 9 for SEQ ID NOs:6 and 7) to link biotin phosphoramidite to the nucleic acid.

TABLE 1
Arm Sequences of Various HSP Designs

| HSP | First | | Second | |
|---|---|---|---|---|
| Name | Arm Sequence | SEQ ID | Arm Sequence | SEQ ID |
| 14-12 | ACGCTGAACTGC | NO:1 | CAGTACGCTGAACT | NO:5 |
| 14-11 | CGCTGAACTGC | NO:2 | CAGTACGCTGAACT | NO:5 |
| 14-13 | TACGCTGAACTGC | NO:3 | CAGTACGCTGAACT | NO:5 |
| 15-13 | TACGCTGAACTGC | NO:3 | CAGTACGCTGAACTG | NO:6 |
| 16-12 | ACGCTGAACTGC | NO:1 | CAGTACGCTGAACTGC | NO:7 |
| 16-13 | TACGCTGAACTGC | NO:3 | CAGTACGCTGAACTGC | NO:7 |
| 16-14 | GTACGCTGAACTGC | NO:4 | CAGTACGCTGAACTGC | NO:7 |

TABLE 2
Sequences of Various HSP Designs

| | | |
|---|---|---|
| HSP | Sequence | SEQ ID |

| | | |
|---|---|---|
| 14-11 | CGCTGAACTGCTTTTTGCAGTTCAGCGTACTGTTTTTCAGTACGCTGAACT | NO:9 |
| 14-12 | ACGCTGAACTGCTTTTTGCAGTTCAGCGTACTGTTTTTCAGTACGCTGAACT | NO:10 |
| 14-13 | TACGCTGAACTGCTTTTTGCAGTTCAGCGTACTGTTTTTCAGTACGCTGAACT | NO:11 |
| 15-13 | TACGCTGAACTGCTTTTTGCAGTTCAGCGTACTGTTTTTCAGTACGCTGAACTG | NO:12 |
| 16-12 | ACGCTGAACTGCTTTTTGCAGTTCAGCGTACTGTTTTTCAGTACGCTGAACTGC | NO:13 |
| 16-13 | TACGCTGAACTGCTTTTTGCAGTTCAGCGTACTGTTTTTCAGTACGCTGAACTG | NO:14 |
| 16-14 | GTACGCTGAACTGCTTTTTGCAGTTCAGCGTACTGTTTTTCAGTACGCTGAACTGC | NO:15 |

In these HSP embodiments, the 3′ second arm sequence is longer than the 5′ first arm sequence so that, in the absence of the analyte, the second arm sequence with its attached biotin is favored to hybridize to the support sequence and form a hybridization duplex, instead of the first arm sequence forming a duplex with the support sequence. When the analyte, streptavidin, is present and binds to the binding pair member, biotin, on the second arm, then the duplex made up of the second arm and the support sequences is destabilized by the streptavidin-biotin binding pair complex, which favors formation of a duplex made up of the first arm and the support sequences. That is, when the specific binding pair complex forms, the HSP switches from a first conformation having a 3′ arm-support sequences duplex, to a second conformation having a 5′ arm-support sequences duplex. The AE label attached to the first arm sequence is relatively protected from hydrolysis by the duplex of the second conformation. If the mixture containing the second conformation (i.e., analyte bound to the HSP) is titrated with biotin, the solution-phase biotin competes with the biotin attached to the HSP for binding to the analyte, streptavidin. The solution-phase biotin may remove streptavidin from the streptavidin-biotin complex attached to the HSP, resulting in a HSP conformational shift, i.e., a switch from the second conformation (analyte-bound HSP) to the first conformation (analyte-free HSP) because a hybridization duplex made up the second arm and support sequences is favored due to the relative length of the second arm sequence compared to the first arm sequence.

These HSPs were tested independently in replicate reactions (3 duplicates per HSP) in aqueous reaction mixtures containing a constant amount of the HSP to be tested, e.g., an amount of AE-labeled HSP to provide about 5×10⁶ RLU per reaction. The reaction mixtures (0.05 ml per assay) each contained a fixed amount of the HSP in hybridization reagent mixed with varying amounts of streptavidin (0 to 250-fold relative to the biotin attached to the HSP) which were incubated for 15 min at room temperature to allow formation of the binding pair complexes (biotin and streptavidin) and the conformational change. Then, the mixtures were mixed with selection reagent (0.25 ml), covered with a layer or inert oil to reduce evaporation, incubated at 60° C. for 10 min to hydrolyze AE not present in a duplex structure, and then cooled to room temperature. Chemiluminescence from the remaining label was initiated and the signal was detected as RLU (2 sec) by using a luminometer (HC LEADER®), substantially as described in U.S. Pat. No. 5,658,737 at column 25, lines 27-46, and Nelson et al., 1996, Biochem. 35:8429-8438 at 8432).

Results of assays performed by using HSP14-13 (SEQ ID NO:11) and HSP15-13 (SEQ ID NO:12) are shown in Table 3 (reported as mean RLU detected in 3 assays per condition). These results show that the presence of the analyte increased the detectable signal significantly for all tests relative to the control that contained no streptavidin.

TABLE 3
Signal Detected for Analyte-Binding Assays Using Two HSPs

| Streptavidin:Biotin | Detected Signal | Detected Signal |
|---|---|---|
| Ratio | for HSP 14-13 | for HSP 15-13 |
| 0:1 | 7.69 × 10⁴ | 6.56 × 10³ |
| 0.5:1 | 1.90 × 10⁵ | 2.10 × 10⁴ |
| 1:1 | 1.96 × 10⁵ | 2.33 × 10⁴ |
| 10:1 | 2.47 × 10⁵ | 2.49 × 10⁴ |
| 25:1 | 2.45 × 10⁵ | 2.67 × 10⁴ |
| 250:1 | 2.51 × 10⁵ | 2.74 × 10⁴ |

In similar experiments, the HSPs were mixed with 0 to 10-fold excess streptavidin in a lithium succinate buffered solution (probe reagent) and then treated to hydrolyze AE not present in a duplex structure and RLU signals were detected using conditions as described above. In those experiments, the detectable signal (RLU) was significantly greater when the analyte was present in the assay mixture than in the control that contained no analyte, but the maximum detected signal was about 100-fold less than the input maximum signal. Therefore, experiments were performed to determine a temperature range in which these HSP-based analyte binding assays were effective and produced optimal signals.

### EXAMPLE 2

### HSP-Based Analyte Detection Assays at Different Temperatures

In this example, assays were performed using conditions similar to those described in Example 1, except that the incubation temperatures in the assays were in a range of 22° C. to 70° C. for the selection step before signal was detected for each assay condition. For these tests, HSP 15-13 (SEQ ID NO:12) was mixed with a 10-fold excess of analyte (0.01 ml of 0.71 mM HSP mixed with 1 ml of 0.071 mM streptavidin) in probe reagent. Controls for each condition contained the same amount of the HSP in the same reagents but without the analyte. For each test, 0.2 ml of the mixture was incubated 15 min at room temperature to allow the solution-phase streptavidin and HSP-attached biotin to form a specific binding pair complex on the HSP. Then each mixture was mixed with 0.25 ml of selection reagent, covered with a layer of inert oil to prevent evaporation, and incubated 30 min at 22° C., 30° C., 37° C, 44° C., 50° C., 60° C., or 70° C. to inactive the AE label attached to substantially single-stranded the HSP. Chemiluminescent signals (RLU) were detected substantially as described in Example 1. Table 4 shows the results obtained, reported as RLU detected for mixtures that contained 10-fold excess streptavidin and control mixtures without streptavidin, and the ratios of the signals detected with and without streptavidin (“detected signal ratio”) for each temperature.

TABLE 4
Assays Performed with HSP 15-13 at Various Selection Temperatures

| Temperature | RLU With | RLU Without | Detected |
|---|---|---|---|
| (° C.) | Streptavidin | Streptavidin | Signal Ratio |
| 22 | 4.51 × 10⁶ | 2.40 × 10⁶ | 1.8 |
| 30 | 3.97 × 10⁶ | 1.07 × 10⁶ | 3.7 |
| 37 | 2.67 × 10⁶ | 2.32 × 10⁵ | 11.5 |
| 44 | 1.16 × 10⁶ | 6.27 × 10⁴ | 18.5 |
| 50 | 2.47 × 10⁵ | 1.36 × 10⁴ | 18.0 |
| 60 | 2.22 × 10³ | 8.34 × 10² | 2.6 |
| 70 | 7.06 × 10² | 7.16 × 10² | 0.98 |

The results shown in Table 4 demonstrate that the HSP-based assay detected the analyte over a temperature range of room temperature to about 60° C., with the highest ratio of signals for samples that contained analyte and compared to controls without analyte observed in the range of about 30° C. to about 50° C., and the greatest signal in analyte-positive tests observed when the selection step was performed in a range of from room temperature to 44° C. These results show that a HSP labeled with a chemiluminescent compound can readily detect analyte by a HSP conformational change that results in protection of the label, which can be detected over at least a 20° C. temperature range.

Similar experiments were performed using HSP 15-13 with attached biotin, incubated with or without the streptavidin analyte, to determine whether a HSP-based assay that includes AE hydrolysis for the detection step functions under additional conditions. In these tests, AE hydrolysis was performed at 37° C. for 5 min to 90 min, by using a selection reagent having a pH of 9.0, 9.3 or 9.6 (pH of the selection reagent was adjusted by addition of NaOH). All of these conditions resulted in detectable signal for mixtures that contained the analyte compared to control assays performed identically on mixtures without analyte (background signal), but assays performed using the pH 9.6 selection reagent gave the best signal to background ratio. These results show that HSP-based assays function under a variety of conditions.

### EXAMPLE 3

### Titration of Analyte in HSP-based Assays Using HSP 15-13 and HSP 16-14

This example shows the sensitivity of an HSP assay by titrating the analyte in HSP-based assays. In these tests, a constant amount of AE-labeled HSP 15-13 (SEQ ID NO:12) with attached biotin was used (40 fmol per assay) and the amount of analyte (streptavidin) was varied to achieve a molar ratio of the analyte and the binding pair member attached to the HSP of 1×10⁻⁵, 1×10⁻⁴, 1×10⁻³, 1×10⁻², 0.1, 0.25, 0.5, 1, and 10. The negative control assay contained no analyte. The assay was performed substantially as described in Example 2, using hydrolysis conditions of 15 min incubation at 37° C. by using selection reagent at pH 9.6 to selectively hydrolyze the AE label attached to a sequence not present in a hybridization duplex. Ten replicates were tested for each analyte concentration arid the chemiluminescent signals (RLU) were detected as described in Example 1. The mean RLU detected in these tests are shown in Table 5.

TABLE 5
Titration of Analyte Using HSP 15-13

| | Molar Ratio of Analyte/ | |
|---|---|---|
| Steptavidin (fmol) | Binding Partner on HSP | Detected Signal |
| 400 | 10 | 1.74 × 10⁶ |
| 40 | 1 | 1.58 × 10⁶ |
| 20 | 0.5 | 1.45 × 10⁶ |
| 10 | 0.25 | 8.27 × 10⁵ |
| 4 | 0.1 | 4.23 × 10⁵ |
| 0.4 | 1 × 10⁻² | 1.28 × 10⁵ |
| 0.04 | 1 × 10⁻³ | 1.02 × 10⁵ |
| 0.004 | 1 × 10⁻⁴ | 9.51 × 10⁴ |
| 0.0004 | 1 × 10⁻⁵ | 9.76 × 10⁴ |
| 0 (Control) | 0 | 8.36 × 10⁴ |

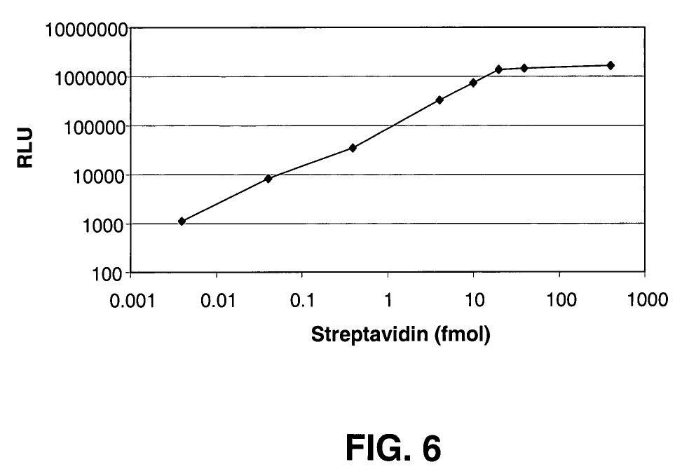

The background signal (RLU from a control reaction containing no analyte) was subtracted from the results of analyte-positive samples and the results are graphically shown in the titration curve of FIG. 6. These results shows that the HSP-based assay has high sensitivity, detecting 0.01 fmol or more of the analyte, and detects the analyte over a broad dynamic range, from 0.01 fmol to 40 fmol.

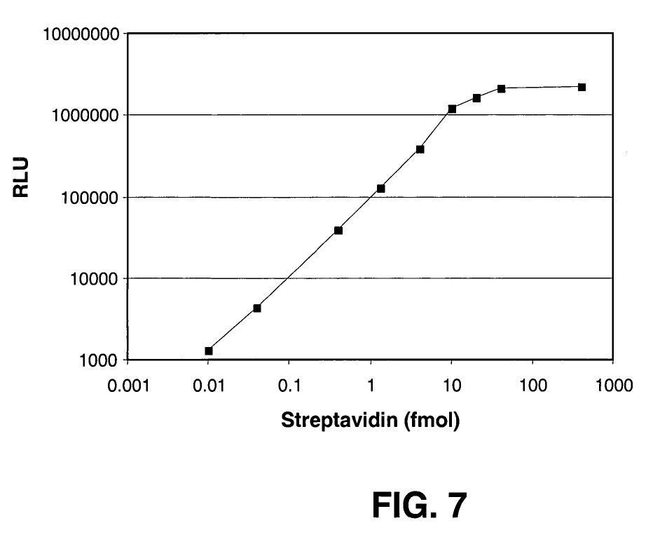

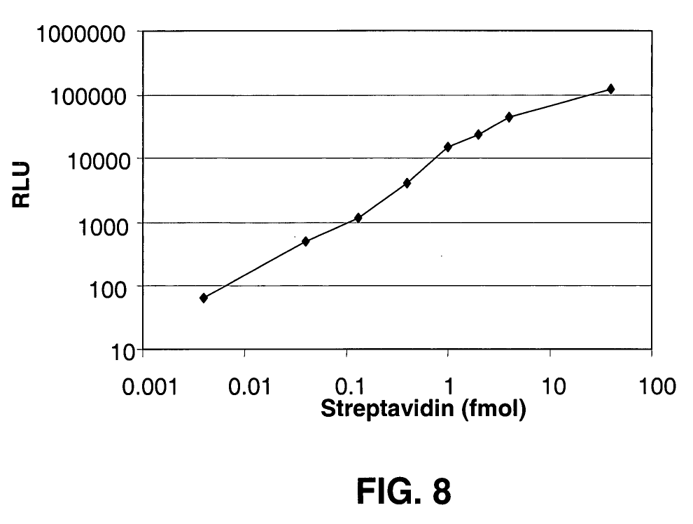

Similar titration assays were performed using a constant amount (40 fmol) of AE-labeled HSP 16-14 (SEQ ID NO:15) with attached biotin by using varying amounts (0 to 1000 fmol) of the analyte, streptavidin. The results of those tests, with the background signal subtracted, also produced a similar titration curve as shown in FIG. 7. Additional assays were performed using a lower concentration (2 fmol) of HSP 16-14 and a lower concentration (0 to 100 fmol) of streptavidin. The results of those tests with the background signal subtracted are graphically shown in the titration curve of FIG. 8. All of these results demonstrate the high sensitivity and broad dynamic range of HSP-based detection assays.

### EXAMPLE 4

### HSP Embodiments with Longer Arm Sequences

For comparison to the HSPs and HSP-assays described in the previous examples, four additional HSPs were designed with two base length differences between the first and second arm sequences. These HSPs were referred to as HSPs 17-15, 18-16, 19-17, and 20-18. The first and second arm sequences of these HSPs are shown in Table 6 (SEQ ID Nos. 16-23). The support sequences were SEQ ID NO:24 for HSP 17-15 and HSP 18-16, and SEQ ID NO:25 for HSP 19-17 and HSP 20-18. The HSPs were synthesized by using standard chemical reactions to link the arm and support sequences in the 5′ to 3′ order arm 1-support-arm 2, joined by linker elements made up of a short homopolymer sequence (T₅), as shown by the sequences in Table 7 (SEQ ID Nos. 26 to 29), with arm sequences in italics and support sequences underlined. For each HSP, both of arm sequences can hybridize to a sequence in the support sequence. In the HSP oligomers, the second arm sequences (SEQ ID Nos. 20-23) had a covalently attached biotin by using biotin phosphoramidite and a chemical linkage between residues 9 and 10 for SEQ ID Nos. 20 and 21, and residues 10 and 11 for SEQ ID Nos. 22 and 23. The HSP oligomers were labeled with an AE compound on the first arm sequences (SEQ ID Nos. 16 to 19), attached by using a covalent chemical linkage between residues 8 and 9 for SEQ ID NO:16, residues 9 and 10 for SEQ ID Nos. 17 and 18, and residues 10 and 11 for SEQ ID NO:19, substantially as described in Example 1.

TABLE 6
Arm Sequences of Various HSPs

| | | | | |
|---|---|---|---|---|
| HSP Name | First Arm Sequence | SEQ ID | Second Arm Sequence | SEQ ID |

| | | | | |
|---|---|---|---|---|
| 17-15 | GTACGCTGAACTGCG | NO:16 | GCAGTACGCTGAACTGC | NO:20 |
| 18-16 | AGTACGCTGAACTGCG | NO:17 | GCAGTACGCTGAACTGCG | NO:21 |
| 19-17 | AGTACGCTGAACTGCGT | NO:18 | TGCAGTACGCTGAACTGCG | NO:22 |
| 20-18 | CAGTACGCTGAACTGCGT | NO:19 | TGCAGTACGCTGAACTGCGT | NO:23 |

TABLE 7
Sequences of Longer HSPs

| | | |
|---|---|---|
| HSP | Sequence | SEQ ID |

| | | |
|---|---|---|
| 17-15 | GTACGCTGAACTGCGTTTTTCGCAGTTCAGCGTACTGCTTTTTGCAGTACGCTGA | NO:26 |
| | ACTGC | |
| 18-16 | AGTACGCTGAACTGCGTTTTTCGCAGTTCAGCGTACTGCTTTTTGCAGTACGCTG | NO:27 |
| | AACTGCG | |
| 19-17 | AGTACGCTGAACTGCGTTTTTTACGCAGTTCAGCGTACTGCATTTTTTGCAGTACG | NO:28 |
| | CTGAACTGCG | |
| 20-18 | CAGTACGCTGAACTGCGTTTTTTACGCAGTTCAGCGTACTGCATTTTTTGCAGTAC | NO:29 |
| | GCTGAACTGCGT | |

HSP-based assays were performed using these longer HSPs with the analyte (streptavidin) and the conformational change in the HSPs in the presence of the analyte was detected by measuring chemiluminescence following hydrolysis of the AE label, using methods substantially as described in Examples 1 to 3. For comparison to these longer HSPs, HSPs 14-12, 15-13, and 16-14 (described in Example 1) were simultaneously tested using the same procedures. Briefly, a 0.1 ml solution containing the HSP oligomer and the streptavidin analyte was mixed with 0.1 ml of a lithium succinate buffered probe reagent, the mixture was incubated at room temperature 10-15 min, then 0.25 ml of selection reagent (pH 9.6) was added, and the mixture was incubated at 37° C. for AE hydrolysis for various times over a 40 min period. After incubation at 37° C. for 0, 5, 10, 15, 20, 30, and 40 min for hydrolysis of AE attached to a sequence not in a hybridization duplex, chemiluminescence was detected as described in Example 1. Control mixtures without streptavidin were treated identically for each HSP.

For all of the HSPs tested, the detected chemiluminescent signals (RLU) were higher when the analyte streptavidin was present, compared to controls that contained no streptavidin, generally in the time period from 0 to 20 min, as shown by the results in Table 8. For each hydrolysis time (5, 10, 15, 20, 30, and 40 min) the detected signal (“Signal”) and signal to background ratio (“Signal/Bkgd”) are shown for each of HSPs 17-15, 18-16, 19-17 and 20-18. For each HSP, the signal was calculated as the mean RLU detected for 10 replicate samples tested in the presence of analyte minus the mean RLU detected for 10 replicate control samples tested without analyte using the same HSP. The “Signal/Bkgd” ratio was calculated by dividing the mean RLU detected for the 10 replicate samples tested in the presence of analyte by the mean RLU detected for the 10 replicate control samples for the same HSP.

TABLE 8
Detection of Analyte Binding Using HSPs with Longer Arm Sequences

| | Time | | | | | | |
|---|---|---|---|---|---|---|---|
| HSP | | 5 min | 10 min | 15 min | 20 min | 30 min | 40 min |
| 17-15 | Signal | 9.95 × 10⁵ | 4.60 × 10⁵ | 2.99 × 10⁵ | 1.44 × 10⁵ | 4.78 × 10⁴ | 1.86 × 10⁴ |
| | Signal/Bkgd | 21.8 | 29.1 | 31.3 | 26.0 | 17.5 | 10.8 |
| 18-16 | Signal | 6.06 × 10⁵ | 2.38 × 10⁵ | 1.11 × 10⁵ | 6.51 × 10⁴ | 2.02 × 10⁴ | 8.65 × 10³ |
| | Signal/Bkgd | 16.7 | 21.2 | 16.6 | 13.5 | 7.5 | 5.5 |
| 19-17 | Signal | 7.90 × 10⁵ | 3.40 × 10⁵ | 2.01 × 10⁵ | 9.81 × 10⁴ | 2.22 × 10⁴ | 1.01 × 10⁴ |
| | Signal/Bkgd | 23.6 | 27.9 | 23.8 | 19.1 | 10.4 | 7.4 |
| 20-18 | Signal | 9.77 × 10⁵ | 4.97 × 10⁵ | 3.23 × 10⁵ | 1.97 × 10⁵ | 6.06 × 10⁴ | 2.59 × 10⁴ |
| | Signal/Bkgd | 9.0 | 10.8 | 11.3 | 11.6 | 9.6 | 7.7 |

When the detected chemiluminescence results were graphed, all of the hydrolysis curves for analyte-positive samples were similar, but the hydrolysis curves in the absence of the analyte showed faster hydrolysis of the AE labels attached to HSP with longer arms compared to HSP with shorter arms.

### EXAMPLE 5

### HSP-Based Competition Titration Assay

This example presents results obtained in a titration assay that uses similar conditions to those described in Example 3, except that biotin was mixed with streptavidin before the HSP oligomer was added to the assay mixture. Because the solution-phase biotin can bind to streptavidin in the mixture before HSP is added, less analyte is available to bind to the biotin attached to the HSP, thus producing fewer HSP conformational changes detected by measuring chemiluminescence. That is, in the presence of increasing amounts of solution-phase biotin, fewer HSPs change conformation and more of the AE label is present in unhybridized strands (i.e., not protected in a hybridization duplex), resulting in more AE hydrolysis and a decrease in detectable signal in the assay.

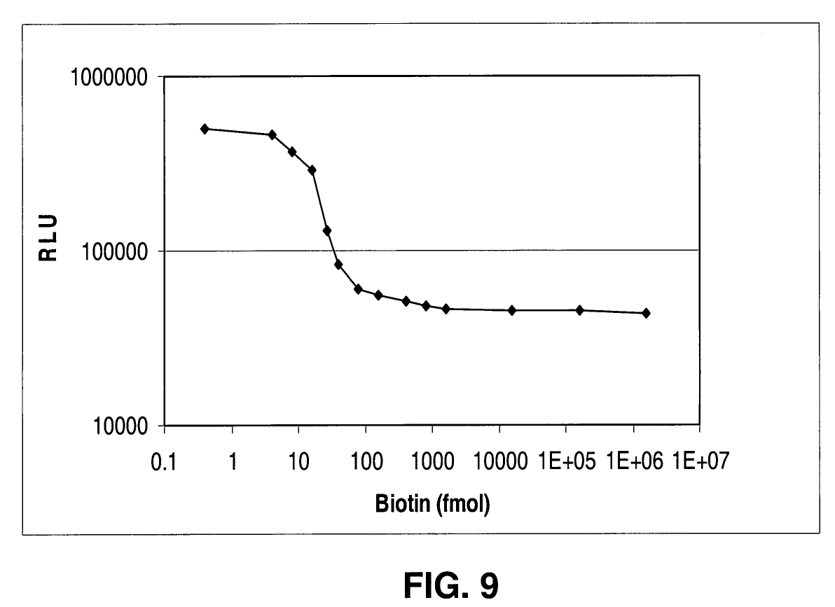

In the assay, substantially the same procedure as described in Example 3 was used, except that the first mixture was an aqueous mixture of streptavidin (9 fmol) and biotin (0 to 10⁷ fmol), and then the AE-labeled HSP 16-14 with biotin attached to an arm sequence (40 fmol) was added with probe reagent. Following incubation of the mixture at room temperature for 10-15 min to allow the available streptavidin and HSP-attached biotin to form a specific binding pair complex on the HSP, hydrolysis of the AE label on unhybridized arm sequences was performed and the chemiluminescent signal was detected as described above. The results of this competition titration assay are shown graphically in FIG. 9, showing the biotin amounts (fmol) present in the reaction mixture on the X-axis and the detected signal (RLU) on the Y-axis. These results show that when less than 10 fmol of competitor biotin was present, the HSP changed its conformation due to analyte binding to the HSP which was detected by the relatively high signal (5×10⁵ RLU or greater). With increasing amounts of competitor biotin present in the mixture, less analyte was available to bind the biotin on the HSP, resulting in fewer HSPs switching to a second conformation, indicated by the decreasing detected signal. The results show that the solution-phase biotin competes with the biotin attached to the HSP for the analyte, streptavidin, resulting in a HSP conformation in which the AE-labeled arm sequence is not in a hybridization duplex, the AE label is not protected from hydrolysis, and the signal decreases.

### EXAMPLE 6

### Analyte Detection Using Fluorophore-Labeled HSPs

This example demonstrates that a HSP labeled with a fluorophore detects analyte in a HSP-based assay in which analyte binding to the HSP changes the HSP conformation that is detected by detecting fluorescence associated with the HSP conformational change. HSPs described in this example have an attached fluorophore (fluorescein) at an internal position adjacent to a linker element (a poly-T sequence) located immediately after the 5′ arm sequence and before the support sequence, and a quencher compound attached at the end of the 3′ arm sequence that has an attached biotin moiety that serves as the binding pair member to detect the analyte. When the analyte, streptavidin, is absent the HSP is in a first conformation in which the quencher and fluorophore are in close proximity due to a hybridization duplex formed between the biotin-attached arm sequence and the support sequence, resulting in little fluorescence emitted from the fluorophore. When the analyte binds to the biotin moiety of the HSP, the duplex is destabilized and the HSP shifts to a second conformation, resulting in separation of the fluorescein label and the quencher, thus producing increased detectable fluorescence from the fluorophore.

Fluorophore-labeled HSPs were designed and synthesized. One probe, fluorescent HSP16-14 (SEQ ID NO:15), was synthesized with an internal fluorescein label, an attached biotin binding pair member, and a 3′ quencher compound (Dabcyl or “Dab”). This synthetic oligomer is shown schematically with the nucleotide sequences and relative positions of the non-nucleic acid moieties as: 5′ GTACGCTGACTGCTTTTT-(Fluorescein)-GCAGTTCAGCGTACTGTTTTTCAGTACGC-(Biotin)-TGAACTGC-(Dab) 3′. Another probe, fluorescent HSP 20-18 (SEQ ID NO:29), was synthesized with an internal fluorescein label, an attached biotin binding pair member, and a 3′ quencher compound (BH2), shown schematically with the nucleotide sequences and relative positions of non-nucleic acid moieties as:

5′ CAGTACGCTGAACTGCGTTTTTT-(Fluorescein)-ACGCAGTTC
AGCGTACTGCATTTTTTGCAGTACGC-(Biotin)-TGAACTGCGT-
(BH2) 3′.

Additional fluorophore-labeled HSPs with attached biotin were designed (SEQ ID Nos. 30-32) to contain a sequence capable of forming a hairpin conformation by hybridization duplex formation involving in the 3′ and 5′ sequences which a poly-T sequence forming the loop of the hairpin, where the loop length was 5, 10 or 15 nucleotides long. These HSPs are similar to the embodiment illustrated in FIG. 1B and referred to as HSP6, HSP6-10, and HSP6-15, are shown schematically with the nucleotide sequences and relative positions of non-nucleic acid moieties as:

| | |
|---|---|
| 5′ (Fluorescein)-CCGAG- | (HSP6, SEQ ID NO:30) |
| (Biotin)-TTTTTTACTCGG- | |
| (Dab) 3′, | |
| 5′ (Fluorescein)-CCGAG- | (HSP6-10, SEQ ID NO:31) |
| (Biotin)-TTTTTTTTTTTACTCGG | |
| -(Dab) 3′, | |
| and | |
| 5′ (Fluorescein)-CCGAG- | (HSP6-15, SEQ ID NO:32) |
| (Biotin)-TTTTTTTTTTTTTTTT | |
| ACTCGG-(Dab) 3′. | |

Fluorophore-labeled HSPs were tested in assays to detect binding of the analyte streptavidin to the biotin binding partner attached to the HSP, using methods similar to those described in Examples 1 to 5 except that the selection and chemiluminescence steps were eliminated, and a fluorescent signal was detected by using a fluorometer. Briefly, after mixing of the fluorophore-labeled HSP (2 pmol) with varying amounts of streptavidin (in 0.01 to 100-fold molar amounts relative to the HSP-attached biotin) in conditions that allow binding of the streptavidin to the HSP-attached biotin (e.g., 15 min at 37° C. in probe reagent), the reaction mixtures were analyzed for fluorescent emission using a device that detects fluorescence in the appropriate wavelength for the fluorophore (for fluorescein, using 470 nm as the excitation wavelength and detecting emission at 510 nm for 4 sec). Controls contained the same reaction components except no streptavidin and were treated using the same steps and conditions as the experimental samples. The tests were performed using an automated device to detect fluorescence (ROTOR-GENE™ 3000, Corbett Robotics Inc., San Francisco, Calif.), although other automated formats or manual steps may be used to perform the assays. Three replicate assays were performed for each assay condition and the mean fluorescence intensity calculated. Experiments performed with fluorophore-labeled HSP6 did not give an increased signal even with 100-fold excess streptavidin, suggesting that streptavidin-biotin binding did not occur or the binding occurred but did not result in a conformational change in HSP6. In contrast, the binding assays performed with fluorophore-labeled HSP 6-10 and HSP 6-15 showed similar increased fluorescence when incubated in the presence of the analyte, streptavidin. Results obtained using fluorescein-labeled HSPs 16-14 and 6-10 are shown in Table 9.

TABLE 9
HSP-based Assay Using Fluorescein-labeled HSPs

Fluorescence Intensity

| Streptavidin:Biotin Ratio | HSP 16-14 | HSP 6-10 |
|---|---|---|
| 100:1 | 41.5 | 31.2 |
| 10:1 | 40.7 | 30.3 |
| 2:1 | 32.4 | 16.7 |
| 1:1 | 26.7 | 12.4 |
| 05.:1 | 14.8 | 6.8 |
| 0.25:1 | 11.6 | 4.4 |
| 0.1:1 | 5.3 | 2.8 |
| 0.01:1 | 2.5 | 2.9 |
| 0 (control) | 2.4 | 3.0 |

The results shown in Table 9 show that a HSP labeled with a fluorescent compound can detect binding of the analyte for the binding partner attached to the HSP arm sequence by detecting an increase in fluorescence proportional to the amount of analyte in the sample. With increasing amounts of the analyte, streptavidin, an increase in fluorescent signal was detected indicating that binding of the analyte to the biotin moiety of the HSP destabilized the hybridization duplex that held the fluorophore and the quencher compounds into close proximity in the first HSP conformation. With release of the duplex involving the biotin-associated arm sequence the HSP changed to a second conformation that resulted in increased detectable signal.

### EXAMPLE 7

### Detection of Protein Analytes in HSP-Based Assays

This example describes methods to detect protein analytes in samples derived from tissues, namely prions present in cell lysates made from mammalian tissue. Tissue samples (e.g., brain and/or spinal cord) are obtained from animals exhibiting symptoms of transmissible spongiform encephalopathy (TSE) diseases, such as from cows with symptoms of bovine spongiform encephalopathy (BSE) and red deer with symptoms of chronic wasting disease. The tissue samples are physically disrupted and cells are lysed by using standard laboratory practices (e.g., minced tissue subjected to detergent lysis). The lysate is treated with nucleases (e.g., DNase and RNase) to limit sample viscosity and destroy nucleic acids, resulting in a protein extract sample that is used for detection of prions (proteinaceous infectious particles, or PrP^{SC}) in HSP-based assays.

A HSP oligonucleotide (similar to the HSP shown in FIG. 1D) is synthesized having the elements, in the order, a 5′ first arm sequence of about 18 nt with an attached AE label, a first linker element (LE), a second arm sequence of about 20 nt, a second LE that includes an aptamer that binds PrP^{SC} but does not bind the corresponding normal cellular protein (PrP^{C}), and a 3′ support sequence of about 20 nt that can hybridize independently to sequences in the first arm sequence and the second arm sequence. Under nucleic acid hybridizing conditions in the absence of PrP^{SC}, the second arm preferentially hybridizes to the support sequence forming a duplex and leaving the AE-labeled first arm sequence single stranded in the first HSP conformation. In the presence of PrP^{SC}, the aptamer binds the PrP^{SC}, causing a conformational change in the HSP that dissociates the duplex made up of the second arm and the support sequences, allowing hybridization of the first arm and the support sequences, resulting in the second HSP conformation. In the first HSP conformation, the AE label is susceptible to hydrolysis, but in the second HSP conformation, the AE label is protected from hydrolysis in conditions previously described in detail (U.S. Pat. Nos. 5,283,174 and 5,639,604). This HSP in hybridization reagent (i.e., in the first HSP conformation) is mixed in individual reaction mixtures with the protein extract samples described above and incubated at about 22° C. to 45° C. for 10-20 min to allow formation of the binding pair complex made up of the PrP^{SC} and the aptamer of the second LE, resulting in formation of the second HSP conformation. Then the reaction mixtures are treated substantially as described in Example 2 to hydrolyze the AE label in single-stranded first arm sequences using conditions that provide an optimal signal to background ratio (e.g., about 44° C. to 50° C.) and the chemiluminescent signals are detected for each assay. As controls, normal tissue samples are obtained from animals that do not exhibit symptoms of a TSE disease and have had no known contact with animals having a TSE disease, from which protein extract samples are prepared and tested using the same procedures and reagents as used for testing the TSE-associated samples. As a background control, the HSP is treated under the same conditions as described above except that no protein extract sample is included in the assay. Detection of chemiluminescence (RLU) that is significantly above background indicates that the HSP has switched from the first to the second conformation, which indicates the presence of PrP^{SC} in the tested sample. None of the normal tissue extract samples produce chemiluminescence that is significantly above the background level, but about 5-10% of the extract samples from animals exhibiting TSE symptoms produce chemiluminescence that is significantly above the background level and significantly above the level of the normal control assays, indicating the presence of PrP^{SC} in the TSE-associated samples that produce elevated chemiluminescence in the HSP-based assays.

### EXAMPLE 8

### Detection of Protein Analytes in HSP-Based Assays

This example tests samples as described in Example 7, but uses a HSP that does not include the AE label and, instead, uses a portion of the HSP sequence that participates in a nucleic acid amplification step to produce detectable amplified nucleic acids. Thus, an amplified signal is produced from HSPs in the conformation that indicates the presence of the analyte in the sample.

A HSP oligonucleotide is synthesized having the elements, in the order, a 5′ first arm sequence of about 30 nt that includes an aptamer that binds PrP^{SC}but does not bind PrP^{C}, a first linker element (LE) that is a single-stranded DNA sequence that includes a promoter sequence, a support sequence of about 20 nt that can hybridize independently to sequences in the first arm sequence and a second arm sequence, a second LE, and the 3′ second arm sequence of about 18 nt. For example, the promoter sequence is a bacteriophage T7 promoter sequence that is recognized by T7 RNA polymerase when the promoter sequence is double stranded. Under nucleic acid hybridizing conditions in the absence of PrP^{SC}, the first arm preferentially hybridizes to the support sequence forming a duplex and leaving the second arm sequence single stranded in the first HSP conformation. In the presence of PrP^{SC}, the aptamer in the first arm sequence binds the PrP^{SC}, causing a conformational change that dissociates the duplex made up of the first arm and support sequences, allowing hybridization of the second arm and the support sequences, resulting in the second HSP conformation. In the first HSP conformation, the 3′ end of the HSP oligonucleotide is present on the single-stranded second arm that cannot serve as a primer for polymerization of nucleic acid because no template strand is associated with the 3′ end of the HSP. In the second HSP conformation, the 3′ end of the HSP oligonucleotide is present in the duplex made up of the second arm and support sequences, and therefore the 3′ end can serve as a primer for polymerization of nucleic acid using a portion of the support sequence, the first LE and the first arm sequences as a template strand.

This HSP in the first conformation (i.e., in hybridizing conditions without PrP^{SC} present) is mixed in individual reaction mixtures with the protein extracts described in Example 7 and incubated at 22° C. to 45° C. for 10-20 min to allow formation of the binding pair complex made up of the PrP^{SC} and the aptamer of the first arm, thus destabilizing the duplex made up of the first arm and support sequences. This allows formation of a duplex made up of the second arm and support sequences, i.e., a switch to the second HSP conformation. Then the reaction mixtures are mixed with a reverse transcriptase (RT) enzyme (e.g., MMLV RT), dNTP substrates and appropriate salts and buffers to allow DNA synthesis to proceed from the 3′ end of the HSP. That is, the second arm sequence serves as a primer for nucleic acid synthesis using as the template strand a portion of the support sequence, the first LE and first arm sequences, to produce a double-stranded functional promoter. The reactions are mixed with the appropriate RNA polymerase for the promoter (e.g., T7 RNA polymerase), rNTP substrates and appropriate salts and buffers to allow RNA synthesis (transcription) to proceed from the functional promoter, making multiple copies (transcripts) of nucleic acid sequences contained in the HSP. These amplified copies or transcripts are detected by using any standard method (e.g., by using a dye or labeled hybridization probe), which produces an amplified signal that indicates the second HSP conformation formed due to the presence of PrP^{SC} in the sample.

In a similar assay, the same steps are performed as described above and then the transcripts are further amplified in a subsequent nucleic acid amplification step. That is, the transcripts serve as templates in a further amplification reaction, such as a transcription mediated amplification (TMA) (U.S. Pat. Nos. 5,399,491, 5,480,784, 5,824,518 and 5,888,779, Kacian et al.), a NASBA reaction (U.S. Pat. No. 5,130, 238, Malek et al., U.S. Pat. No. 5,409,818, Davey et al.), or a polymerase chain reaction (PCR) using the RT supplied in the earlier reaction mixture (U.S. Pat. Nos. 4,683,195, 4,683,202, and 4,800,159, Mullis et al.). The additional amplified sequences produced in those TMA, NASBA or PCR reactions are detected by using any standard method (e.g., a dye or labeled hybridization probe) to produce an amplified signal that indicates the HSP changed to the second HSP conformation due to the presence of the PrP^{SC} analyte in the tested sample.

Using these methods, an amplified signal that is significantly greater than the signal from assays using the normal control samples is detected for about 30-90% of the protein extract samples from animals exhibiting TSE symptoms. The increased signals indicate the presence of PrP^{SC} in some of the TSE-associated samples and show the increased sensitivity of HSP-based assays that include a signal amplification step.

The foregoing examples illustrate some embodiments of the invention, although other embodiments are encompassed by the claims that follow.

We claim:

1. A hybridization switch probe (HSP) specific for detection of an analyte, comprising:
a first nucleic acid arm sequence;
a second nucleic acid arm sequence that is different from the first nucleic acid arm sequence;
a nucleic acid support sequence that is at least partially complementary to the first nucleic acid arm sequence and at least partially complementary to the second nucleic acid arm sequence, whereby under hybridization conditions the support sequence forms a hybridization duplex with either
the first nucleic acid arm sequence thereby forming a first HSP conformation, or
the second nucleic acid arm sequence thereby forming a second HSP conformation;
a label that produces a signal that indicates the conformation of the hybridization switch probe,
and a binding pair member that forms a specific binding pair complex with the analyte, wherein the specific binding pair complex produces a conformational change in the hybridization switch probe that results in a detectable signal.

2. The hybridization switch probe of claim 1, wherein the first arm sequence is shorter than the second arm sequence.

3. The hybridization switch probe of claim 1, wherein the label produces a signal that is detectable in a homogeneous assay system.

4. The hybridization switch probe of claim 1, wherein the label is a portion of the HSP nucleic acid.

5. The hybridization switch probe of claim 1, wherein the label is a separate moiety joined directly or indirectly to the HSP.

6. The hybridization switch probe of claim 4, wherein the label is selected from the group consisting of:
a HSP nucleic acid sequence that binds a separate nucleic acid probe sequence,
a HSP nucleic acid sequence that serves as a primer in a nucleic acid amplification reaction,
a HSP nucleic acid sequence that serves as a template in a nucleic acid amplification reaction, and an aptamer.

7. The hybridization switch probe of claim 5, wherein the label is selected from the group consisting of a radionuclide, a ligand, an enzyme, an enzyme substrate, an enzyme cofactor, a reactive group, a chromophore, a particle, a bioluminescent compound, a phosphorescent compound, a chemiluminescent compound, and a fluorophore.

8. The hybridization switch probe of claim 1, wherein the label is a chemiluminescent compound attached to either the first arm sequence or the second arm sequence.

9. The hybridization switch probe of claim 1, wherein
the label is a fluorophore attached to the first arm sequence and the support sequence includes a quencher compound that is in close proximity to the fluorophore when the first arm sequence and the support sequence form a hybridization duplex, or
the label is a fluorophore attached to the second arm sequence and the support sequence includes a quencher compound that is in close proximity to the fluorophore when the second arm sequence and the support sequence form a hybridization duplex, or
the label is a fluorophore attached to the support sequence and the first arm sequence includes a quencher compound that is in close proximity to the fluorophore when the first arm sequence and the support sequence form a hybridization duplex, or
the label is a fluorophore attached to the support sequence and the second arm sequence includes a quencher compound that is in close proximity to the fluorophore when the second arm sequence and the support sequence form a hybridization duplex.

10. The hybridization switch probe of claim 1, wherein the first arm sequence is joined to the support sequence by a linking element and the second arm sequence is joined to the support sequence by a linking element.

11. The hybridization switch probe of claim 1, wherein the binding pair member that forms a specific binding pair complex with the analyte is an aptamer.

12. The hybridization switch probe of claim 1, wherein the detectable signal is an amplified nucleic acid that is produced by use of a portion of the HSP participating in a nucleic acid amplification reaction.

13. A kit comprising a hybridization switch probe that comprises:
a first nucleic acid arm sequence;
a second nucleic acid arm sequence that is different from the first nucleic acid arm sequence;
a nucleic acid support sequence that is at least partially complementary to the first nucleic acid arm sequence and to the second nucleic acid arm sequence, whereby under hybridization conditions the support sequence forms a hybridization duplex with the first nucleic acid arm sequence to form a first conformation of the hybridization switch probe, or with the second nucleic acid arm sequence to form a second conformation of the hybridization switch probe;
a label that produces a signal that indicates the conformation of the hybridization switch probe;
and a binding pair member that forms a specific binding pair complex with an analyte detected by the hybridization switch probe, wherein the specific binding pair complex produces a conformational change in the hybridization switch probe that results in a detectable signal from the label.

14. A kit of claim 13, further comprising one or more reagents for preparation of a sample containing the analyte, one or more reagents that promote binding of the analyte and the binding pair member, one or more reagents that treat the label to produce a detectable signal, or one or more reagents used in a nucleic acid amplification reaction that amplifies a nucleic acid sequence by using a portion of the HSP sequence.

15. A method of detecting an analyte in a sample, comprising:
forming a reaction mixture comprising a sample containing an analyte and a hybridization switch probe specific for the analyte,
wherein the hybridization switch probe is made up of a first nucleic acid arm sequence, a second nucleic acid arm sequence that is different from the first nucleic acid arm sequence, a nucleic acid support sequence that is at least partially complementary to the first nucleic acid arm sequence and to the second nucleic acid arm sequence, a label that produces a detectable signal, and a binding pair member that binds the analyte to form a specific binding pair complex that produces a conformational change in the hybridization switch probe, and
wherein the hybridization switch probe is in a first HSP conformation in which one arm sequence is in a hybridization duplex with the support sequence;
binding the analyte to the binding pair member, thereby forming a specific binding pair complex on the hybridization switch probe;
producing a conformational change from the first HSP conformation to a second HSP conformation resulting from formation of the specific binding pair complex; and
detecting a signal change from the label that indicates the conformational change, thereby indicating the presence of the analyte in the sample.

16. The method of claim 15, wherein the first arm sequence of the hybridization switch probe has an attached label, the second arm sequence has an attached binding pair member, and the first HSP conformation includes a hybridization duplex made up of the second arm sequence and the support sequence which is destabilized when the specific binding pair complex is formed, thereby changing the hybridization switch probe to the second HSP conformation that includes a hybridization duplex made up of the first arm sequence and the support sequence.

17. The method of claim 15, wherein the second arm sequence of the hybridization switch probe has an attached label, the first arm sequence has an attached binding pair member, and the first HSP conformation includes a hybridization duplex made up of the first arm sequence and the support sequence which is destabilized when the specific binding pair complex is formed, thereby changing the hybridization switch probe to the second HSP conformation that includes a hybridization duplex made up of the second arm sequence and the support sequence.

18. The method of claim 15, wherein the one arm sequence of the hybridization switch probe is a labeled arm sequence that has both an attached label and an attached binding pair member, and the first HSP conformation includes a hybridization duplex made up of the labeled arm sequence and the support sequence which is destabilized when the specific binding pair complex is formed, thereby changing the hybridization switch probe to the second HSP conformation in which the labeled arm sequence is not hybridized to the support sequence.

19. The method of claim 15, wherein the analyte is a ligand that binds specifically to the binding pair member and both the binding pair member and the analyte are known chemical or biochemical structures.

20. The method of claim 15, wherein the analyte is a ligand that binds specifically to the binding pair member and either the ligand or the binding pair member has an unknown chemical or biochemical structure.

21. The method of claim 15, wherein the binding pair member is a portion of a nucleic acid sequence in the hybridization switch probe.

22. The method of claim 15, wherein the binding pair member is an aptamer.

23. The method of claim 15, wherein the detecting step detects an increase in a detectable signal to indicate the presence of the analyte in the sample.

24. The method of claim 15, wherein the detecting step detects a decrease in a detectable signal to indicate the presence of the analyte in the sample.

25. The method of claim 15, wherein the detecting step detects a signal resulting from in vitro amplification of a nucleic acid sequence present in the hybridization switch probe.

26. The method of claim 15, wherein the detecting step detects a signal resulting from using a portion of the hybridization switch probe in the second HSP conformation as a primer in an in vitro nucleic acid amplification reaction.

27. The method of claim 15, wherein the detecting step detects a signal resulting from using a portion of the hybridization switch probe in the second HSP conformation as a template in an in vitro nucleic acid amplification reaction.

28. The method of claim 15, wherein the detecting step detects a signal resulting from using a portion of the hybridization switch probe in the first HSP conformation as a primer in an in vitro nucleic acid amplification reaction.

29. The method of claim 15, wherein the detecting step detects a signal resulting from using a portion of the hybridization switch probe in the first HSP conformation as a template in an in vitro nucleic acid amplification reaction.

30. The method of claim 15, wherein the detecting step detects a signal resulting from in vitro amplification of a sequence that is only amplified when the hybridization switch probe is in the second HSP conformation.

31. The method of claim 15, wherein the detecting step detects a signal resulting from in vitro amplification of a sequence that is only amplified when the hybridization switch probe is in the first HSP conformation.

32. The method of claim 15, wherein the detecting step is performed in a homogeneous format.


SEQUENCE LISTING (32 sequences)

SEQ ID NO: 1 - DNA, 12 bp, Artificial
  Synthetic oligonucleotide
acgctgaactgc

SEQ ID NO: 2 - DNA, 11 bp, Artificial
  Synthetic oligonucleotide
cgctgaactgc

SEQ ID NO: 3 - DNA, 13 bp, Artificial
  Synthetic oligonucleotide
tacgctgaactgc

SEQ ID NO: 4 - DNA, 14 bp, Artificial
  Synthetic oligonucleotide
gtacgctgaactgc

SEQ ID NO: 5 - DNA, 14 bp, Artificial
  Synthetic oligonucleotide
cagtacgctgaact

SEQ ID NO: 6 - DNA, 15 bp, Artificial
  Synthetic oligonucleotide
cagtacgctgaactg

SEQ ID NO: 7 - DNA, 16 bp, Artificial
  Synthetic oligonucleotide
cagtacgctgaactgc

SEQ ID NO: 8 - DNA, 16 bp, Artificial
  Synthetic oligonucleotide
gcagttcagcgtactg

SEQ ID NO: 9 - DNA, 51 bp, Artificial
  Synthetic oligomer
cgctgaactgctttttgcagttcagcgtactgtttttcagtacgctgaact

SEQ ID NO: 10 - DNA, 52 bp, Artificial
  Synthetic oligonucleotide
acgctgaactgctttttgcagttcagcgtactgtttttcagtacgctgaact

SEQ ID NO: 11 - DNA, 53 bp, Artificial
  Synthetic oligonucleotide
tacgctgaactgctttttgcagttcagcgtactgtttttcagtacgctgaact

SEQ ID NO: 12 - DNA, 54 bp, Artificial
  Synthetic oligonucleotide
tacgctgaactgctttttgcagttcagcgtactgtttttcagtacgctgaactg

SEQ ID NO: 13 - DNA, 54 bp, Artificial
  Synthetic oligonucleotide
acgctgaactgctttttgcagttcagcgtactgtttttcagtacgctgaactgc

SEQ ID NO: 14 - DNA, 55 bp, Artificial
  Synthetic oligonucleotide
tacgctgaactgctttttgcagttcagcgtactgtttttcagtacgctgaactgc

SEQ ID NO: 15 - DNA, 56 bp, Artificial
  Synthetic oligonucleotide
gtacgctgaactgctttttgcagttcagcgtactgtttttcagtacgctgaactgc

SEQ ID NO: 16 - DNA, 15 bp, Artificial
  Synthetic oligonucleotide
gtacgctgaactgcg

SEQ ID NO: 17 - DNA, 16 bp, Artificial
  Synthetic oligonucleotide
agtacgctgaactgcg

SEQ ID NO: 18 - DNA, 17 bp, Artificial
  Synthetic oligonucleotide
agtacgctgaactgcgt

SEQ ID NO: 19 - DNA, 18 bp, Artificial
  Synthetic oligonucleotide
cagtacgctgaactgcgt

SEQ ID NO: 20 - DNA, 17 bp, Artificial
  Synthetic oligonucleotide
gcagtacgctgaactgc

SEQ ID NO: 21 - DNA, 18 bp, Artificial
  Synthetic oligonucleotide
gcagtacgctgaactgcg

SEQ ID NO: 22 - DNA, 19 bp, Artificial
  Synthetic oligonucleotide
tgcagtacgctgaactgcg

SEQ ID NO: 23 - DNA, 20 bp, Artificial
  Synthetic oligomer
tgcagtacgctgaactgcgt

SEQ ID NO: 24 - DNA, 18 bp, Artificial
  Synthetic oligomer
cgcagttcagcgtactgc

SEQ ID NO: 25 - DNA, 20 bp, Artificial
  Synthetic oligonucleotide
acgcagttcagcgtactgca

SEQ ID NO: 26 - DNA, 60 bp, Artificial
  Synthetic oligonucleotide
gtacgctgaactgcgtttttcgcagttcagcgtactgctttttgcagtacgctgaactgc

SEQ ID NO: 27 - DNA, 62 bp, Artificial
  Synthetic oligonucleotide
agtacgctgaactgcgtttttcgcagttcagcgtactgctttttgcagtacgctgaactgcg

SEQ ID NO: 28 - DNA, 66 bp, Artificial
  Synthetic oligonucleotide
agtacgctgaactgcgttttttacgcagttcagcgtactgcattttttgcagtacgctgaactgcg

SEQ ID NO: 29 - DNA, 68 bp, Artificial
  Synthetic oligonucleotide
cagtacgctgaactgcgttttttacgcagttcagcgtactgcattttttgcagtacgctgaactgcgt

SEQ ID NO: 30 - DNA, 17 bp, Artificial
  Synthetic oligonucleotide
ccgagttttttactcgg

SEQ ID NO: 31 - DNA, 22 bp, Artificial
  Synthetic oligonucleotide
ccgagtttttttttttactcgg

SEQ ID NO: 32 - DNA, 27 bp, Artificial
  Synthetic oligonucleotide
ccgagttttttttttttttttactcgg